(19) United States
(12) Patent Application Publication
(10) Pub. No.: US 2015/0301067 A1
(43) Pub. Date: Oct. 22, 2015

(54) METHODS AND ASSAYS FOR FACIOSCAPULOHUMERAL MUSCULAR DYSTROPHY

(71) Applicant: UNIVERSITY OF WASHINGTON THROUGH ITS CENTER FOR COMMERCIALIZATION, Seattle, WA (US)

(72) Inventors: Daniel G. Miller, Seattle, WA (US); Gregory J. Block, Seattle, WA (US)

(73) Assignee: UNIVERSITY OF WASHINGTON THROUGH ITS CENTER FOR COMMERCIALIZATION, Seattle, WA (US)

(21) Appl. No.: 14/439,924

(22) PCT Filed: Nov. 5, 2013
(86) PCT No.: PCT/US2013/068391

§ 371 Date: Apr. 30, 2015

Related U.S. Application Data
(60) Provisional application No. 61/722,420, filed on Nov. 5, 2012.

(51) Int. Cl. G01N 33/68; G01N 33/50; C12N 5/077

(52) U.S. Cl. CPC G01N 33/6893; C12N 5/0658; G01N 33/5061; G01N 33/5023; C12N 2500/90; C12N 2500/30; C12N 2500/36; C12N 2506/1323; G01N 2333/47; G01N 2800/10

(57) ABSTRACT

Provided herein are methods, assays and compositions relating to the treatment of FSHD, particularly by modulating expression of DUX4.

### CROSS-REFERENCE TO RELATED APPLICATIONS

This application claims benefit under 35 U.S.C. §119(e) of U.S. Provisional Application No. 61/722,420, filed Nov. 5, 2012, the content of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Nov. 5, 2013, is named 034186-079330_SL.txt and is 2,325 bytes in size.

### TECHNICAL FIELD

The invention relates to the treatment of facioscapulohumeral muscular dystrophy and to screening assays for identification of agents useful for the same.

### BACKGROUND

Facioscapulohumeral Muscular dystrophy (FSHD) is a dominantly inherited disease characterized by progressive weakening of select skeletal muscle groups¹. FSHD is believed to be caused by chromatin relaxation at the D4Z4 macrosatellite array on chromosome 42-4 allowing transcription of the normally repressed DUX4 gene located within each D4Z4 units^{5 6-9}. The chromatin relaxation occurs in FSHD1 by truncation of the array to less than 11 units⁴ or in FSHD2 by a contraction-independent mechanism¹⁰ caused by mutations in SMCHD1¹¹. No animal models exist and cultured myoblasts from FSHD-muscle biopsies contain DUX4 in 0.1% of nuclei and grow and differentiate normally¹²⁻¹⁵ making it difficult to identify pathways that regulate DUX4 and influence disease progression¹⁶.

### SUMMARY

The methods and assays described herein are based, in part, on the discovery that muscle cells from FSHD patients which do not express DUX4 or an FSHD phenotype under standard culture conditions can be induced to express the FSHD phenotype, including expression of DUX4 in vitro. Thus, provided herein are drug screening assays for identifying candidate agents for the treatment of DUX4. Also provided herein are methods and culture conditions for inducing a muscle cell obtained from an FSHD patient to express DUX4, wherein the methods and culture conditions do not induce a muscle cell from a control subject to express DUX4.

Provided herein in one aspect is a method for inducing skeletal muscle cells from an individual with Facioscapulohumeral Muscular Dystrophy (FSHD) to express an FSHD phenotype in culture, the method comprising contacting said cells with a serum-free medium.

In one embodiment of this aspect and all other aspects described herein, the serum-free medium comprises a serum-free stem cell medium. In some embodiments, the serum-free stem cell medium is a stem cell maintenance medium.

In another embodiment of this aspect and all other aspects described herein, the medium comprises a serum replacement composition.

In another embodiment of this aspect and all other aspects described herein, the serum replacement composition comprises a lipid-rich albumin fraction.

In another embodiment of this aspect and all other aspects described herein, the lipid-rich albumin fraction comprises a bovine serum albumin preparation or a human serum albumin preparation.

In another embodiment of this aspect and all other aspects described herein, the serum-free medium comprises one or more amino acids, one or more antioxidants, one or more hormones, or one or more trace element compositions.

In another embodiment of this aspect and all other aspects described herein, the method further comprises measuring the expression of DUX4 in said cells.

In another embodiment of this aspect and all other aspects described herein, the cells assume a myotube morphology.

Also provided herein in another aspect is a screening assay comprising: (a) culturing a cell or population of cells obtained from a subject having, or at risk of having, FSHD under conditions that permit DUX4 expression, (b) contacting the cell or population of cells with an agent, and (c) measuring DUX-4 expression in the cell or population of cells, wherein a decrease in the expression of DUX4 indicates that the agent is a candidate agent for treating FSHD.

In another embodiment of this aspect and all other aspects described herein, the cells assume a myotube morphology.

In another embodiment of this aspect and all other aspects described herein, the conditions that permit DUX4 expression comprise culture in serum-free medium comprising a serum replacement composition. In one embodiment of this aspect and all other aspects described herein, the serum-free medium comprises a serum-free stem cell medium. In some embodiments, the serum-free stem cell medium is a stem cell maintenance medium.

In another embodiment of this aspect and all other aspects described herein, the serum replacement composition comprises a lipid-rich albumin fraction.

In another embodiment of this aspect and all other aspects described herein, the lipid-rich albumin fraction comprises bovine serum albumin or human serum albumin.

In another embodiment of this aspect and all other aspects described herein, the candidate agent reduces the amount of cytopathic lesions, apoptosis, and/or retracted myotubes in the population of cells as compared to a substantially identical cell population cultured under the same conditions but in the absence of the candidate agent.

Also provided herein are uses of a serum-free medium for inducing in vitro expression of DUX4 in muscle cells obtained from a subject having, or at risk of having, FSHD.

In one embodiment of this aspect and all other aspects described herein, the serum-free medium comprises a serum-free stem cell medium. In some embodiments, the serum-free stem cell medium is a stem cell maintenance medium.

In another embodiment of this aspect and all other aspects described herein, the serum-free stem cell medium comprises a serum replacement composition.

In another embodiment of this aspect and all other aspects described herein, the serum replacement composition comprises a lipid-rich albumin fraction.

In another embodiment of this aspect and all other aspects described herein, the lipid-rich albumin fraction comprises bovine serum albumin.

In another embodiment of this aspect and all other aspects described herein, the lipid-rich albumin fraction comprises human serum albumin.

Another aspect provided herein relates to a method for treating FSHD in a subject, the method comprising: administering an inhibitor of DUX4 expression to a subject having, or at risk of having, FSHD, wherein the inhibitor of DUX4 expression is selected from the group consisting of: an activator of the Wnt/β-catenin pathway, a tankyrase inhibitor, a GSK-3β inhibitor, and an activator of DNMT-1, thereby treating FSHD in the subject.

In another embodiment of this aspect and all other aspects described herein, the activator of the Wnt/β-catenin pathway comprises a recombinant Wnt peptide or polypeptide, or a combination thereof.

In another embodiment of this aspect and all other aspects described herein, the activator of the Wnt/β-catenin pathway comprises a nucleic acid encoding a recombinant Wnt peptide or polypeptide, or a combination thereof.

In another embodiment of this aspect and all other aspects described herein, the recombinant Wnt peptide is Wnt3a or Wnt9B.

In another embodiment of this aspect and all other aspects described herein, the tankyrase inhibitor comprises Wiki4, XAV-939, IWR, or JW55.

In another embodiment of this aspect and all other aspects described herein, the method further comprises a step of diagnosing the subject with FSHD.

### BRIEF DESCRIPTION OF THE FIGURES

FIGS. 1A-1D show Knockout Serum Replacement (KOSR)-containing differentiation medium generates hypertrophic myotubes that express DUX4. FIG. 1A is a bar graph showing the average width of MHC (+) myotubes (*=p<0.05 by two-tailed Student's T-Test) determined using immunofluorescent microscopy of non-FSHD primary human myoblasts (2081) differentiated by mitogen depletion using HS/ITS or KOSR. Nuclei were counterstained with DAPI and myotubes were stained for myosin heavy chain (MHC) (data not shown). FIG. 1B is a scatter plot displaying the number of nuclei per cluster demonstrating that myotubes differentiated in KOSR medium contain large clusters that contain more nuclei. A cluster of nuclei is a grouping of nuclei adjacent to one another inside a myotube. p<0.05 by Mann-Whitney test for non-parametric distribution. FIG. 1C shows the percentage of total nuclei positive for DUX4 immunofluorescence within MHC(+) myotubes for two non-FSHD and three FSHD-derived myoblasts differentiated in HS/ITS or KOSR containing medium. Results are pooled between the three cell lines (FSHD1(2082), FSHD2(2305), and FSHD2(1881). p<0.05 based on Fisher Exact Test. FIG. 1D shows data for quantitative PCR of DUX4-activated genes, MDB2L3 and CCNA1 in three FSHD-derived cell lines (FSHD1(2082), FSHD2(2305), FSHD2(1881)). Data are displayed as the change in expression level when myoblasts are differentiated in KOSR compared to HS/ITS. *=p<0.05 by Student's T-test.

FIGS. 2A-2E show that myoblasts from FSHD-muscle biopsies undergo DUX4-dependent death when differentiated to myotubes. FIG. 2A shows FSHD myoblasts that were transfected with non-targeting or DUX4-targeting siRNAs and differentiated into myotubes. 96 hrs post-differentiation, dishes were stained with antibodies to MHC and the myotube fusion index was calculated. p<0.05 by Student's T-test. FIG. 2B shows the average number of contracted MHC(+) myotubes containing clusters of pyknotic nuclei per well of a 384 well dish 96 hrs following transfection of the DUX4-targeting siRNA. p<0.05 by Student's T-test. FIG. 2C Since apoptotic myotubes were non-adherent, conditioned medium from myotubes transfected with non-targeting or DUX4-targeting siRNA was harvested and incubated with DAPI to measure DNA concentration in the conditioned medium. p<0.05 by Student's T-test. FIG. 2D Average number of contracted MHC(+) myotubes immunoreactive for cleaved caspase-3 96 hrs following transfection of the DUX4-targeting siRNA. p<0.05 by Student's T-test. FIG. 2E FSHD1(2349) myotubes were differentiated in KOSR medium containing 10 μM of the ROCK inhibitor Y-27632 or 0.5 μM Thiazovivin and immunostained for DUX4 and MHC. DUX4/MHC(+) nuclei were counted for each condition. *=p<0.05 by analysis of variance.

FIGS. 3A-3F Activation of the Wnt/β-catenin signaling pathway reduces DUX4 expression in FSHD myotubes. FIG. 3A C2C12 cells containing an integrated luciferase gene with the DUX4 promoter (D4Z4(FFL)) were cultured in myoblast proliferation medium or differentiation medium in the presence or absence of 3 μM GSK inhibitor CHIR99021 (GSKi). Luciferase activity was normalized to DNA content. Shown is the fold change in Luciferase activity when compared to cells treated with DMSO vehicle control. p<0.05 by Student's T-test. FIG. 3B Upper: schematic diagram showing the relative positions of primers to amplify DUX4 transcripts. The DUX4 open reading frame is shown as a yellow box with two black boxes representing the homeobox domains. The polyadenylation signal downstream of the open reading frame is shown as a black box. A thin black line below the open reading frame shows the splice products generated. The direction and approximate location of the primers is shown with black arrows. Lower: RT PCR of non-FSHD and FSHD-derived cells cultured in myoblast proliferation or differentiation medium in the absence or presence of GSK inhibitor. Transcription of the Wnt target gene, Axin2, was monitored as a control for activation of the Wnt/β-catenin signaling. FIG. 3C Counts of DUX4(+) nuclei in MHC(+) myotubes shown in panel c derived from three separate cell lines. p<0.05 by Student's T-Test. FIG. 3D shows myotube index of experiments where primary FSHD(2349) myoblasts were differentiated in medium containing GSKi or 250 ng/ml recombinant Wnt3A (rWnt3A) or relevant vehicle controls (DMSO for GSKi or 0.1% CHAPS for rhWnt3A). Myotubes were fixed at 48 hrs to quantify DUX4 protein expression, and 72 hrs to measure myotube loss. p<0.05 by Student's T-Test. FIG. 3E shows the quantification of DUX4(+) nuclei from experiments using immunofluorescent microscopy assay for DUX4 expression in myotubes transfected with an siRNA targeting CTNNB1 (the gene encoding f3-catenin (siCTNNB1); p<0.05 by Student's T-test FIG. 3F shows results from quantitative PCR amplification for the transcripts of DUX4-activated genes CCNA1 and MBD3L2 from FSHD2(2305) transfected with non-targeting or β-catenin targeting siRNA. p<0.05 by Student's T-Test.

FIGS. 4A-4D DUX4 expression increases when SMCHD1 levels are reduced resulting in a shorter time to apoptosis in FSHD1 myotubes. FIG. 4A shows counts of DUX4(+) nuclei in experiments from two primary FSHD1 cell lines (2349 and 2088) that were transduced with a lentivirus vector encoding a non-targeting or SMCHD1-targeting shRNA. The cells were differentiated into myotubes and immunostained for DUX4 and MHC at 48 hrs post-differentiation (2088 line shown). p<0.05 by Students T-Test. FIG. 4B shows DNMT1 transcript quantification by quantitative PCR of RNA from non-FSHD myoblasts (2401) transduced with a lentivirus containing a non-targeting or DNMT1-targeting shRNA. Cells were stained for DUX4 and counterstained with DAPI (data not shown). *=p<0.05 by Students T-Test. FIG. 4C Transcript quantification of DUX4-activated genes CCNA1 and MBD3L2 by quantitative PCR of RNA from cells prepared as in e. Data are displayed as fold increase over transcript levels in the same cells transduced with a non-targeted shRNA control vector. * p<0.05 by Students T-Test. FIG. 4D shows quantification of DUX4 expression in FSHD1(2349) and FSHD(2088). FSHD1 myoblasts (2081) were transduced with a lentivirus containing a non-targeting or DNMT1-targeting shRNA. Cells were stained for DUX4, MHC and counterstained with DAPI (data not shown).

FIG. 5 is a table summarizing the cell lines used in the study. FSHD and non-FSHD myotubes were scored for DUX4-associated cell death. The size of the chromosome 4 D4Z4 array with an A161 haplotype is displayed in kb and the estimated number of D4Z4 repeats is shown. The relative amounts of DUX4 detected in the cultures following differentiation in KOSR medium are represented by asterisks, X if no DUX4 was detected. *=<1%, **=1%>10%, ***>10% DUX4(+) nuclei. The cell lines were obtained with IRB approval from individuals with contraction dependent (FSHD1), contraction independent (FSHD2), or from non-affected individuals (Non-FSHD). DUX4-associated myotube apoptosis was detected in all cell lines that underwent efficient differentiation in KOSR medium, meaning that DUX4(+) nuclei were adjacent to cytopathic lesions. The column “myoblasts” indicates the number of MHC(−) nuclei expressing DUX4 in non-differentiated cultures of primary myoblasts.

FIG. 6 is a bar graph showing the average number of cytopathic lesions in culture dishes of 3 different cells lines differentiated in KOSR vs. HS/ITS differentiation medium.

FIGS. 7A-7B Optimization of DUX4 knockdown. FIG. 7A FSHD myoblasts were transfected with two siRNAs targeting DUX4 (DUX4-1 and DUX4-4). Twenty-four hrs later, the medium was changed to KOSR differentiation medium and 48 hrs later harvested and assayed for DUX4 transcripts by RT-PCR. FIG. 7B Cells prepared as in FIG. 7A, were assayed by qRT-PCR for expression of the two DUX4-activated genes, MBD3L2 and CCNA1.

FIG. 8 Confirmation of reduction of FRG1 transcripts. RT-PCR for FRG1 transcripts following transfection of myotubes with a non-targeting or FRG1-targeting siRNA.

FIG. 9 Treatment of FSHD myotubes with GSKi is insufficient to prevent apoptosis. ATP content of myoblasts or myotubes cultured in the absence or presence of GSKi. No difference was seen between conditions.

FIGS. 10A-10D Reduction of SMCHD1 transcripts in FSHD1 myoblasts results in increased expression of DUX4 and DUX4-activated genes. FIG. 10A FSHD1(2349) and FSHD1(2088) myoblasts were transduced with lentivirus vectors encoding a non-targeting shRNA (shSCR) and an shRNA targeting SMCHD1 (shSMCHD1). Cells were harvested and assayed for SMCHD1 expression by qRT-PCR. FIG. 10B FSHD1(2088) myoblasts differentiated in KOSR medium for 48 hrs and assayed for DUX4 expression by RT-PCR. GAPDH served as an expression control. FIG. 10C Fold change in expression of DUX4-activated genes CCNA1 and MBD3L2 in cells prepared in FIG. 10B. FIG. 10D DUX4 activated gene (CCNA1 and MBD3L2) transcript quantification by quantitative PCR of RNA from cells transduced with lentivirus vectors containing a non-targeting (shSCR) or DNMT1-targeting (shDNMT1) shRNA.

FIG. 11 The genes listed in FIG. 5 were identified in Geng et al 2010 as direct targets of DUX4. It was hypothesized that one or more of these genes is involved in initiating myotube-specific apoptosis in FSHD. The inventors designed an siRNA library containing each of these genes and screened for myotube viability. Of the 85 genes tested, 3 hits were obtained: PRR4, RBBP6, and TAF4B. PRR4 is a secreted protein normally expressed in glandular tissues such as the lacrimal gland of the eye or salivary glands. Although the mechanism by which PRR4 induces myotube-specific apoptosis is unknown, blocking the protein with dominant negative isoforms, specific neutralizing antibodies or small molecules as well as using antisense oligonucleotides, morpholinos, siRNA, miRNA or shRNAs to decrease the amount of PRR4 transcripts are all contemplated interventions for therapy. RBBP6 is a binding partner of p53 and Rb proteins and is known to be involved in apoptosis and cell cycle arrest. TAF4B is a testes-specific transcription initiation factor. Similarly to PRR4, blocking either protein with dominant negative isoforms or small molecules as well as using antisense oligonucleotides, morpholinos, siRNA, miRNA or shRNAs to decrease the amount of transcripts are all contemplated interventions for therapy.

FIG. 12: Primary FSHD myoblasts were transfected with siRNAs corresponding to the list in FIG. 11. Cells were fixed 120 hrs post-differentiation and assayed by immunohistochemistry. The total number of nuclei in myotubes per well was calculated using a pre-defined algorithm. Peaks correspond to “hits” that were consistent across 4 experiments, and are labeled with an arrow.

### DETAILED DESCRIPTION

The methods and assays described herein are based, in part, on the discovery that myotubes from an FSHD patient, which do not express DUX4 or an FSHD phenotype under standard culture conditions, can be cultured in vitro under conditions that induce an FSHD phenotype, including expression of DUX4. The same culture conditions do not induce DUX4 expression to the same degree in myotubes from a non-FSHD subject. Thus, provided herein are screening assays using FSHD myotube cultures to test for candidate agents that reduce DUX4 expression. Also provided herein are novel treatments for FSHD identified using the cultures described herein.

### DEFINITIONS

All scientific and technical terms used in this application have meanings commonly used in the art unless otherwise specified. As used in this application, the following words or phrases have the meanings specified.

As used herein the term “FSHD phenotype” refers to a skeletal muscle cell (e.g., a myotube) that, at a minimum, expresses endogenous DUX4 in culture conditions as described herein. Cell cultures having an FSHD phenotype can also exhibit cytopathic lesions, increased apoptosis, and retracted or contracted myotubes as compared to a skeletal muscle cell having a non-FSHD phenotype.

As used herein, the term “serum free medium” refers to a mammalian cell culture medium that does not contain serum, such as bovine serum (FBS), horse serum, goat serum, human serum, etc.

As used herein the term “serum replacement composition” refers to a composition comprising ingredients to aid in cell survival and/or differentiation and/or growth in the absence of serum, for example, amino acids, antioxidants, iron containing compounds (e.g., transferrin), selenium containing compounds (e.g., sodium selenite), hormones, growth factors, osmotic factors, and/or trace mineral supplying compounds. A serum replacement composition as the term is used herein will include free fatty acids, triacylglycerides, lysophosphatidylcholine, phosphatidylcholine, phosphatidic acid, cholesterol, and/or sphingomyelin, preferably as part of a lipid-rich albumin fraction (e.g., ALBUMAX™, ALBUMAX II, among others). Exemplary amino acids for use in a serum replacement composition include e.g., glycine, L-histidine, L-isoleucine, L-methionine, L-phenylalanine, L-proline, L-hydroxyproline, L-serine, L-threonine L-tryptophan, L-tyrosine, L-valine and/or thiamine. Some non-limiting examples of trace mineral supplying compounds include AgNO₃AlCl₃.6H₂0, Ba(C₂H₃O₂)₂, CdS0₄.8H₂0, CoCl₂.6H₂0, Cr₂(SO₄)₃.1H₂0, GeO₂, Na₂SeO₃, H₂SeO₃, KBr, KI, MnCl₂.4H₂O, NaF, Na₂SiO₃.9H₂O, NaVO₃, (NH₄)₆MO₇O₂₄.4H₂0, NiSo₄.6H₂O, RbCl, SnCl₂, and ZrOCl₂.8H₂0.

As used herein, the term “lipid-rich albumin fraction” refers to a composition comprising albumin (e.g., purified albumin) complexed with lipids including, for example, fatty acids, fat soluble vitamins, etc. At a minimum, a lipid-rich albumin fraction when added as a component of a serum-replacement composition, will promote an FSHD phenotype in cultured skeletal muscle cells from an individual with FSHD.

As used herein, the term “a myotube morphology” refers to a developing muscle fiber cell with a centrally located nucleus, typically generated by fusion of at least two myoblast cells.

As used herein, the term “under conditions that permit DUX4 expression” refers to cell culture conditions in which a proportion of cells obtained from an FSHD subject are induced to express DUX4. These same culture conditions do not induce cells from a non-FSHD subject to express DUX4 to the same degree as cells from an FSHD subject cultured under the same conditions. In some embodiments, the term “under conditions that permit DUX4 expression” refers to cell culture conditions that induce DUX4 expression in at least 3% of myotubes (e.g., when normalized to nuclei number), at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 25%, at least 30%, at least 40%, at least 50%, at least 60%, at least 70%, at least 80%, at least 90%, at least 95%, at least 99% or even 100% of myotubes. In contrast, the “conditions that permit DUX4 expression” will induce DUX4 expression in less than 2% of myotubes obtained from a non-FSHD subject.

“Nucleic acid” refers to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences and as well as the sequence explicitly indicated.

As used herein, “pharmaceutically acceptable salt” refers to a salt that retains the desired biological activity of the parent compound and does not impart any undesired toxicological effects. Examples of such salts include, but are not limited to, (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, furmaric acid, gluconic acid, citric acid, malic acid, ascorbic acid, benzoic acid, tannic acid, pamoic acid, alginic acid, polyglutamic acid, naphthalenesulfonic acids, naphthalenedisulfonic acids, polygalacturonic acid; (b) salts with polyvalent metal cations such as zinc, calcium, bismuth, barium, magnesium, aluminum, copper, cobalt, nickel, cadmium, and the like; or (c) salts formed with an organic cation formed from N,N′-dibenzylethylenediamine or ethylenediamine; or (d) combinations of (a) and (b) or (c), e.g., a zinc tannate salt; and the like. The preferred acid addition salts are the trifluoroacetate salt and the acetate salt.

The term “pharmaceutically acceptable” refers to compounds and compositions which can be administered to mammals without undue toxicity. The term “pharmaceutically acceptable carriers” excludes tissue culture medium.

As used herein, “pharmaceutically acceptable carrier” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline or normal (0.9%) saline. Compositions comprising such carriers are formulated by well-known conventional methods (see, for example, Remington's Pharmaceutical Sciences, 18th edition, A. Gennaro, ed., Mack Publishing Co., Easton, Pa., 1990).

As used herein, “a” or “an” means at least one, unless clearly indicated otherwise. As used herein, to “prevent” or “protect against” a condition or disease means to hinder, reduce or delay the onset or progression of the condition or disease.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g., the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, at least about a 20-fold increase, at least about a 50-fold increase, at least about a 100-fold increase, at least about a 1000-fold increase or more as compared to a reference level.

The term “statistically significant” or “significantly” refers to statistical significance and generally means two standard deviations (2SD) or more above or below normal or a reference. The term refers to statistical evidence that there is a difference. It is defined as the probability of making a decision to reject the null hypothesis when the null hypothesis is actually true. The decision is often made using the p-value.

As used herein, the term “comprising” means that other elements can also be present in addition to the defined elements presented. The use of “comprising” indicates inclusion rather than limitation.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Further, unless otherwise required by context, singular terms shall include pluralities and plural terms shall include the singular.

Other than in the operating examples, or where otherwise indicated, all numbers expressing quantities of ingredients or reaction conditions used herein should be understood as modified in all instances by the term “about.” The term “about” when used in connection with percentages can mean±1%.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

### Facioscapulohumeral Muscular Dystrophy

Facioscapulohumeral muscular dystrophy (FSHD) is an autosomally dominant inherited and progressive disease that preferentially affects muscles of the face (facio), shoulder (scapula), and upper arms (humeral). Although it is primarily a disorder of skeletal muscle, FSHD is also associated with abnormalities of the retinal vasculature, sensorineural hearing defects and, in 12% of cases, cardiac arrhythmias (Am J Med Genet. 1985; 22:143-147; Acta Otolaryngol Suppl. 1995; 520 Pt 1:140-142; Muscle Nerve. 1995; 2:S73-S80; Eur Neurol. 2006; 56:1-5). With an estimated incidence of 1:15,000-1:20,000, there are ˜15,000-20,000 people in the United States of America, and close to 400,000 people worldwide suffering from FSHD. The normal life expectancy, the autosomal dominant mode of inheritance, and the high frequency of new mutations (^{˜}10%) in FSHD patients all contribute to its rank as the third most common form of muscular dystrophy (Muscle Nerve. 2006; 34:1-15).

FSHD is thought to be genetically linked to deletions of integral numbers of 3.3 kb polymorphic repeating sequences from a tandem array, termed D4Z4, at chromosomal position 4q35. Detection of 10 or fewer repeats on chromosome 4 is diagnostic of the disease in over 95% of FSHD cases, but an additional requirement for disease pathogenesis is the inheritance of the 4qA allele on chromosome 4, indicating that a short D4Z4 array alone is not sufficient to cause FSHD (Nat. Genet. 2002; 32:235-236; Am J Hum Genet. 2004; 75:1124-1130).

Symptoms of FSHD can include, but are not limited to, facial muscle weakness (eyelid drooping, inability to whistle, decreased facial expression, depressed or angry facial expression, difficulty pronouncing the letters M, B, and P, shoulder weakness (difficulty working with the arms raised, sloping shoulder), hearing loss, abnormal heart rhythm, unequal weakening of the biceps, triceps, deltoids, and lower arm muscles, loss of strength in abdominal muscles (causing a protuberant abdomen and lumbar lordosis) and eventual progression to the legs, and foot drop (a gait abnormality in which the dropping of the forefoot happens due to weakness, damage to the peroneal nerve or paralysis of the muscles in the anterior portion of the lower leg).

FSHD can be diagnosed by measuring the size of D4Z4 deletions of chromosome 4q35; however other clinical means can also be used to yield a diagnosis of FSHD. Such clinical tests include, but are not limited to, measurement of creatine kinase levels, electromyogram, measurement of nerve conduction velocity, and muscle biopsy. These and other such clinical tests for diagnosing muscular dystrophy are known to those of ordinary skill in the art.

Obtaining and Culturing Muscle Cells from a Subject

As used herein, the term “biological sample” refers to a sample obtained from a subject, and typically comprises at least one muscle cell (e.g., a myoblast, a myocyte, a myotube). In one embodiment, the biological sample is obtained using a muscle biopsy technique, where tissue and cells from a specific muscle are obtained from a subject and can optionally be viewed microscopically. A muscle biopsy procedure requires only a small piece of tissue to be removed from the designated muscle. In one embodiment, the tissue sample is obtained by inserting a biopsy needle into the muscle. If a larger sample is desired, one of skill in the art can perform an open muscle biopsy by e.g., making an incision in the skin and removing a larger section of muscle.

In one embodiment, a biological sample can be obtained from any muscle in the individual to be tested. Exemplary muscle groups include, but are not limited to, the bicep, deltoid, gastrocnemius, soleus, triceps, gluteals, or quadriceps. The muscle selected for the biopsy can be chosen based on the location of symptoms (e.g., pain, weakness, or relevant biomarker).

The muscle cells in the biological sample can be prepared for culture using any of a number of methods known to those of skill in the art including, for example, enzymatic digestion, physical or mechanical disruption, or centrifugation.

### Culture Conditions for Inducing an FSHD Phenotype

Skeletal muscle cells from individuals with FSHD do not generally express an FSHD phenotype under standard muscle cell culture conditions. Provided herein are culture conditions for skeletal muscle cells obtained from a subject having FSHD, wherein the cells are induced to express an FSHD phenotype, including expression of DUX4 in vitro. The culture conditions comprise contacting skeletal muscle cells (e.g., myoblasts) with a serum-free culture medium comprising a serum replacement composition. In one embodiment, the serum-free culture medium comprises an embryonic stem cell medium (e.g., an embryonic stem cell expansion medium). In some embodiments, the culture conditions comprise contacting the cells with free fatty acids, lysophosphatidylcholine, triacylglycerides, phosphatidylcholine, phosphatidic acid, cholesterol and/or sphingomyelin, which can be added directly to the culture medium used herein or can be bound to albumin (e.g., a lipid-rich albumin fraction).

In one embodiment, the serum replacement composition comprises a lipid-rich albumin fraction. In one embodiment, at least 40% of the albumin in a “lipid-rich albumin fraction” is bound to lipids and/or fatty acids. In other embodiments, at least 50%, at least 60%, at least 70%, at least 80%, at least 90%, at least 95%, or at least 99% of the albumin in a lipid-rich albumin fraction is bound to lipids and/or fatty acids and/or lipid soluble molecules (e.g., lipid soluble vitamins) In one embodiment, the lipids and/or fatty acids comprise free fatty acids (FFAs), lysophosphatidylcholine (LPC), triacylglycerides (TAGs), phosphatidylcholine (PC), phosphatidic acid (PA), cholesterol (CH) and/or sphingomyelin (SM). In certain embodiments, the lipid-rich albumin fraction is provided as a 10×, 20×, 50×, or 100× stock solution and is diluted to a 1× solution using the culture medium or serum replacement composition described herein. Alternatively, the lipid-rich albumin fraction is provided as a powder, and can be added to the cell culture medium to make a 1% solution with respect to the lipid-rich albumin fraction. Exemplary final concentrations (e.g., the concentrations in the culture medium in which cells are grown) of lipids and/or fatty acids for the culture conditions contemplated herein are provided below.

In some embodiments, the concentration of free fatty acids in the culture medium described herein is at least 5 μg/mL, at least 10 μg/mL, at least 15 μg/mL, at least 20 μg/mL, at least 25 μg/mL, at least 30 μg/mL, at least 35 μg/mL, at least 40 μg/mL, at least 50 μg/mL or more. The free fatty acids can be added to the medium directly or can be provided as a complex with albumin e.g., in a lipid-rich albumin fraction.

In some embodiments, the concentration of lysophosphatidylcholine in the culture medium is at least 1 μg/mL, at least 2 μg/mL, at least 3 μg/mL, at least 4 μg/mL, at least 5 μg/mL, at least 6 μg/mL, at least 7 μg/mL, at least 8 μg/mL, at least 9 μg/mL, at least 10 μg/mL, at least 11 μg/mL, at least 12 μg/mL, at least 13 μg/mL, at least 14 μg/mL, at least 15 μg/mL or more. Lysophosphatidylcholine can be added to the medium directly or can be provided as a complex with albumin e.g., in a lipid-rich albumin fraction.

In some embodiments, the concentration of triacylglycerides in the culture medium described herein is at least 0.5 μg/mL, at least 1.0 μg/mL, at least 2.0 μg/mL, at least 3.0 μg/mL, at least 4.0 μg/mL, at least 5.0 μg/mL, at least 6.0 μg/mL, at least 7.0 μg/mL, at least 8.0 μg/mL, at least 9.0 μg/mL, at least 9.5 μg/mL, at least 10 μg/mL, at least 11 μg/mL, at least 12 μg/mL or more. The triacylglycerides can be added to the medium directly or can be provided as a complex with albumin e.g., in a lipid-rich albumin fraction.

In some embodiments, the concentration of phosphatidylcholine in the culture medium described herein is at least 0.5 μg/mL, at least 1.0 μg/mL, at least 1.5 μg/mL, at least 2.0 μg/mL, at least 2.5 μg/mL, at least 3.0 μg/mL, at least 3.5 μg/mL, at least 4.0 μg/mL, at least 4.5 μg/mL, at least 5.0 μg/mL, at least 5.5 μg/mL, at least 6.0 μg/mL, at least 6.5 μg/mL, at least 7 μg/mL or more. Phosphatidylcholine can be added to the medium directly or can be provided as a complex with albumin e.g., in a lipid-rich albumin fraction.

In some embodiments, the concentration of phosphatidic acid in the culture medium described herein is at least 0.1 μg/mL, at least 0.25 μg/mL, at least 0.5 μg/mL, at least 0.75 μg/mL, at least 1.0 μg/mL, at least 1.25 μg/mL, at least 1.5 μg/mL, at least 1.75 μg/mL, at least 2.0 μg/mL, at least 2.5 μg/mL, at least 3.0 μg/mL, at least 3.5 μg/mL, at least 4 μg/mL or more. Phosphatidic acid can be added to the medium directly or can be provided as a complex with albumin e.g., in a lipid-rich albumin fraction.

In some embodiments, the concentration of cholesterol in the culture medium described herein is at least 0.05 μg/mL, at least 0.1 μg/mL, at least 0.2 μg/mL, at least 0.3 μg/mL, at least 0.4 μg/mL, at least 0.5 μg/mL, at least 0.6 μg/mL, at least 0.7 μg/mL, at least 0.75 μg/mL, at least 0.8 μg/mL, at least 0.85 μg/mL, at least 0.9 μg/mL, at least 1 μg/mL or more. Cholesterol can be added to the medium directly or can be provided as a complex with albumin e.g., in a lipid-rich albumin fraction.

In some embodiments, the concentration of sphingomyelin in the culture medium described herein is at least 0.05 μg/mL, at least 0.1 μg/mL, at least 0.2 μg/mL, at least 0.3 μg/mL, at least 0.4 μg/mL, at least 0.5 μg/mL, at least 0.6 μg/mL, at least 0.7 μg/mL, at least 0.75 μg/mL, at least 0.8 μg/mL, at least 0.85 μg/mL, at least 0.9 μg/mL, at least 1 μg/mL or more. Sphingomyelin can be added to the medium directly or can be provided as a complex with albumin e.g., in a lipid-rich albumin fraction.

In one embodiment, the lipid-rich albumin fraction is a stock solution that contains 10×, 20×, 50×, or 100× the concentration of one or more components selected from the group consisting of free fatty acids, lysophosphatidylcholine, triacylglycerides, phosphatidylcholine, phosphatidic acid, cholesterol and sphingomyelin as described herein and is diluted to a 1× solution when preparing the culture medium. For example, if the concentration of free fatty acids in the medium is desired to be 30 μg/mL, a 10× stock solution will comprise 300 μg/mL free fatty acids. In another embodiment, the lipid-rich albumin fraction is provided as a powder, from which a skilled artisan can prepare a stock solution of a desired concentration. One of skill in the art will recognize that any number of stock solutions of a lipid-rich fraction can also be prepared. The calculations necessary to yield a concentration of a particular component (e.g., free fatty acids) in the culture medium as described herein are well within the abilities of the ordinary skilled artisan.

In some embodiments, the lipid-rich albumin fraction comprises at least 0.50% lipids and fatty acids by dry weight; in other embodiments the lipids and fatty acids comprise at least 0.55%, at least 0.60%, at least 0.65%, at least 0.7%, at least 0.75% by dry weight of the lipid-rich albumin fraction.

In some embodiments, the lipids in the lipid-rich albumin fraction are comprised of 40-60% free fatty acids, 10-20% lysophosphatidylcholine, 10-20% triacylglycerides, 5-10% phosphatidylcholine, 1-3% phosphatidic acid, ˜1% cholesterol, and ˜1% sphingomyelin. In one embodiment, the lipid-rich albumin fraction comprises a composition as described in e.g., Garcia-Gonzalo PLoS ONE (2008) 3(1):e1384, which is incorporated herein by reference in its entirety.

In some embodiments, a lipid-rich albumin fraction can be obtained commercially, for example, ALBUMAX or ALBUMAX II (LIFE TECHNOLOGIES).

In general, the serum replacement composition is contacted with the e.g., myoblast cells in culture to promote differentiation. As such, it is contemplated herein that the cells are confluent or near confluent at the time the serum replacement composition is added to the culture. In one embodiment, the cells are contacted with the serum replacement composition/medium when the cells are at least 60% confluent, at least 70% confluent, at least 80% confluent, at least 90% confluent, at least 95% confluent, at least 99% confluent or more (e.g., confluent). One of skill in the art will understand what the term “confluent” means but for the avoidance of doubt, 100% confluency means the dish is completely covered by the cells, and therefore no more room is left for the cells to grow; whereas 50 percent confluency means roughly half of the dish is covered and there is still room for cells to grow.

### In Vitro Detection of DUX4 Expression

Provided herein are a variety of assay formats that can be used to determine the presence, concentration or level of DUX4 in a biological sample or a culture of cells derived from a biological sample. Examples of assay formats include known techniques such as Western blot analysis, radioimmunoassay (hereinafter referred to as “RIA”), immunoradiometric assay (IRMA), chemiluminescent immunoassays, such as enzyme-linked immunosorbent assay (hereinafter referred to as “ELISA”), multiplex bead assays, a fluorescence antibody method, passive haemagglutination, mass spectrometry (such as MALDI/TOF (time-of-flight), SELDI/TOF), liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, and tandem mass spectrometry HPLC. Some of the immunoassays can be easily automated by the use of appropriate instruments such as the IM x™ (Abbott, Irving, Tex.) for a fluorescent immunoassay and Ciba Corning ACS 180™ (Ciba Corning, Medfield, Mass.) for a chemiluminescent immunoassay.

RIA and ELISA provide the benefit of detection sensitivity, rapidity, accuracy, possible automation of procedures, and the like, for the determination of the concentration or level of a DUX4 polypeptide or a fragment thereof. Radioimmunoassay (Kashyap, M. L. et al., J. Clin. Invest., 60:171-180 (1977)) is a technique in which a detection antibody can be used after labeling with a radioactive isotope such as ¹²⁵I. Antibody arrays or protein chips can also be employed, see for example U.S. Patent Application Nos: 20030013208A1; 20020155493A1; 20030017515 and U.S. Pat. Nos. 6,329,209; 6,365,418, which are herein incorporated by reference in their entirety.

The most common enzyme immunoassay is ELISA. There are different forms of ELISA which are well known to those skilled in the art, e.g., standard ELISA, competitive ELISA, and sandwich ELISA. The standard techniques for ELISA are described in “Methods in Immunodiagnosis”, 2nd Edition, Rose and Bigazzi, eds. John Wiley & Sons, 1980; Campbell et al., “Methods and Immunology”, W. A. Benjamin, Inc., 1964; and Oellerich, M. 1984, J. Clin. Chem. Clin. Biochem., 22:895-904. ELISA is a technique for detecting and measuring the concentration of an antigen, such as a DUX4 polypeptide or fragment thereof, using a labeled (e.g. enzyme linked) form of the antibody. In a “sandwich ELISA”, an antibody is linked to a solid phase (i.e. a microtiter plate) and exposed to a biological sample containing antigen (e.g. a DUX4 polypeptide or fragment thereof). The solid phase is then washed to remove unbound antigen. A labeled antibody (e.g. enzyme linked) is then bound to the plate bound-antigen (if present) forming an antibody-antigen-antibody sandwich. Examples of enzymes that can be linked to the antibody are alkaline phosphatase, horseradish peroxidase, luciferase, urease, and B-galactosidase. The enzyme linked antibody reacts with a substrate to generate a colored reaction product that can be measured. In a “competitive ELISA”, a specific concentration of an antibody specific for a DUX4 polypeptide or fragment thereof is incubated with a biological sample or cells derived therefrom. The DUX4-antibody mixture is then contacted with a solid phase (e.g. a microtiter plate) that is coated with a DUX4 polypeptide. The more DUX4 polypeptide present in the sample, the less free antibody that will be available to bind to the solid phase. A labeled (e.g., enzyme linked) secondary antibody is then added to the solid phase to determine the amount of primary antibody bound to the solid phase.

### DUX4 Binding Agents and Antibodies

In one embodiment, a binding agent (e.g., a peptide) or antibody that binds to e.g., DUX4 is used in both methods of detecting the presence of amount of DUX4 and in methods and assays for identifying candidate agents for the treatment of FSHD.

An “antibody” that can be used according to the methods described herein includes complete immunoglobulins, antigen binding fragments of immunoglobulins, as well as antigen binding proteins that comprise antigen binding domains of immunoglobulins. Antigen binding fragments of immunoglobulins include, for example, Fab, Fab′, F(ab′)2, scFv and dAbs. Modified antibody formats have been developed which retain binding specificity, but have other characteristics that may be desirable, including for example, bispecificity, multivalence (more than two binding sites), and compact size (e.g., binding domains alone). Single chain antibodies lack some or all of the constant domains of the whole antibodies from which they are derived. Therefore, they can overcome some of the problems associated with the use of whole antibodies. For example, single-chain antibodies tend to be free of certain undesired interactions between heavy-chain constant regions and other biological molecules. Additionally, single-chain antibodies are considerably smaller than whole antibodies and can have greater permeability than whole antibodies, allowing single-chain antibodies to localize and bind to target antigen-binding sites more efficiently. Furthermore, the relatively small size of single-chain antibodies makes them less likely to provoke an unwanted immune response in a recipient than whole antibodies. Antibodies useful in the methods described herein include, but are not limited to, naturally occurring antibodies, bivalent fragments such as (Fab′)2, monovalent fragments such as Fab, single chain antibodies, single chain Fv (scFv), single domain antibodies, multivalent single chain antibodies, diabodies, triabodies, and the like that bind specifically with an antigen.

Antibodies can be raised against a polypeptide or portion of a polypeptide by methods known to those skilled in the art. Antibodies are readily raised in animals such as rabbits or mice by immunization with the gene product, or a fragment thereof. Immunized mice are particularly useful for providing sources of B cells for the manufacture of hybridomas, which in turn are cultured to produce large quantities of monoclonal antibodies. Antibody manufacture methods are described in detail, for example, in Harlow et al., 1988 Antibodies: A Laboratory Manual. Both polyclonal and monoclonal antibodies can be used in the methods described herein. The ordinary skilled artisan is aware of the relative advantages and disadvantages of each type of antibody preparation.

Useful monoclonal antibodies and fragments can be derived from any species (including humans) or can be formed as chimeric proteins which employ sequences from more than one species. If necessary or desired, human monoclonal antibodies or “humanized” murine antibodies can also be used in accordance with the methods and assays described herein.

### Reference Values

The terms “reference value,” “reference level,” “reference sample,” and “reference” are used interchangeably herein and refer to the level of DUX4 expression in a known sample against which another sample (i.e., one obtained from a subject having FSHD) is compared. A reference value is useful for determining the amount of DUX4 expression or the relative increase/decrease of such expression levels in a biological sample or cells derived from the biological sample. A reference value serves as a reference for comparison, such that samples can be normalized to an appropriate standard in order to infer the presence, absence or degree of DUX4 expression in a subject under a given set of circumstances.

In one embodiment, a biological standard is obtained at an earlier time point (e.g., prior to the onset of FSHD) from the same individual that is to be tested or treated as described herein. Alternatively, a standard can be from the same individual having been taken at a time after the onset or diagnosis of FSHD. In such instances, the reference value can provide a measure of the efficacy of treatment. It can be useful to use as a reference for a given patient a level or ratio from a sample taken after diagnosis of FSHD but before the administration of any therapy to that patient.

In one embodiment, a range of values for DUX4 in e.g., a muscle biopsy sample or cells derived from such a sample can be defined for a plurality of individuals with or without detectable FSHD. Provided that the number of individuals in each group is sufficient, one can define a range of DUX4 values for each population. These values can be used to define cut-off points for selecting a therapy or for determining efficacy of a candidate agent. Thus, one of skill in the art can determine the level of DUX4 and compare the value to the ranges in each particular sub-population to aid in determining the status of disease and whether or not a treatment is expected to work in a particular patient. Such value ranges are analogous to e.g., HDL and LDL cholesterol levels detected clinically. For example, LDL levels below 100 mg/dL are considered optimal and do not require therapeutic intervention, while LDL levels above 190 mg/dL are considered ‘very high’ and will likely require some intervention. One of skill in the art can readily define similar parameters for DUX4 expression in a variety of FSHD statuses. These value ranges can be provided to clinicians, for example, on a chart, programmed into a PDA etc.

A standard comprising a reference value or range of values can also be synthesized. A known amount of DUX4 (or a series of known amounts) can be prepared within the typical expression range for DUX4 that is observed in a general FSHD population. In one embodiment, a recombinant DUX4 is used as a standard for generating a reference value or set of values. This method has an advantage of being able to compare the extent of disease in one or more individuals in a mixed population. This method can also be useful for subjects who lack a prior sample to act as a reference value or for routine follow-up post-diagnosis. This type of method can also allow standardized tests to be performed among several clinics, institutions, or countries etc.

### Treatment of FSHD

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder. The term “treating” includes reducing or alleviating at least one adverse effect or symptom of a condition, disease or disorder associated with FSHD. Treatment is generally “effective” if one or more symptoms or clinical markers are reduced. Alternatively, treatment is “effective” if the progression of a disease is reduced or halted. That is, “treatment” includes not just the improvement of symptoms or markers, but can also include a cessation or at least slowing of progress or worsening of symptoms that would be expected in absence of treatment. Beneficial or desired clinical results include, but are not limited to, alleviation of one or more symptom(s) of FSHD (e.g., muscle weakness or atrophy), diminishment of extent of FSHD, stabilized (i.e., not worsening) FSHD, delay or slowing of progression of the disease, amelioration or palliation of the FSHD disease state, and remission (whether partial or total). The term “treatment” of a disease also includes providing at least partial relief from the symptoms or side-effects of the disease (including palliative treatment).

In one embodiment, as used herein, the term “prevention” or “preventing” when used in the context of a subject refers to stopping, hindering, and/or slowing the development of FSHD.

As used herein, the term “therapeutically effective amount” means that amount necessary, at least partly, to attain the desired effect, or to delay the onset of, inhibit the progression of, or halt altogether, the onset or progression of the particular disease or disorder being treated (e.g., FSHD). Such amounts will depend, of course, on the particular condition being treated, the severity of the condition and individual patient parameters including age, physical condition, size, weight and concurrent treatment. These factors are well known to those of ordinary skill in the art and can be addressed with no more than routine experimentation. In some embodiments, a maximum dose of a therapeutic agent is used, that is, the highest safe dose according to sound medical judgment. It will be understood by those of ordinary skill in the art, however, that a lower dose or tolerable dose that is effective can be administered for medical reasons, psychological reasons or for virtually any other reason.

In one embodiment, a therapeutically effective amount of a pharmaceutical formulation, or a composition described herein for a method of treating FSHD is an amount sufficient to reduce the level of at least one symptom of FSHD (e.g., muscle weakness, muscle atrophy etc.) as compared to the level in the absence of the compound, the combination of compounds, the pharmaceutical composition/formulation of the composition. In other embodiments, the amount of the composition administered is preferably safe and sufficient to treat, delay the development of FSHD, and/or delay onset of the disease. In some embodiments, the amount can thus cure or result in amelioration of the symptoms of FSHD, slow the course of the disease, slow or inhibit a symptom of the disease, or slow or inhibit the establishment or development of secondary symptoms of FSHD. As but two examples, an effective amount of a composition described herein can inhibit further symptoms associated with FSHD or cause a reduction in one or more symptoms associated with FSHD. While effective treatment need not necessarily initiate complete regression of the disease, such effect would be effective treatment. The effective amount of a given therapeutic agent will vary with factors such as the nature of the agent, the route of administration, the size and species of the animal to receive the therapeutic agent, and the purpose of the administration. Thus, it is not possible or prudent to specify an exact “therapeutically effective amount.” However, for any given case, an appropriate “effective amount” can be determined by a skilled artisan according to established methods in the art using only routine experimentation.

### Activators of the Wnt/β-Catenin Signaling Pathway

The hallmark of canonical Wnt signaling activation is elevated levels of the protein β-catenin. β-catenin is constitutively produced and is present in the cytoplasm as pools of monomeric protein. (See e.g., Papkoff, J. et al., Mol. Cell Biol. 1996; 16: 2128-2134). The primary mechanism for controlling cytoplasmic levels of β-catenin is through direct physical degradation upon recruitment into a large multi-protein complex (“degradation complex”). After formation, the complex is stabilized by the GSK3β-mediated phosphorylation of the protein components Axin and APC, as well as PP2A. GSK3β-then phosphorylates β-catenin, thereby allowing it to be recognized by β-transducin repeat containing protein (β-TrCP), and targeting it for ubiquitination and proteosomic degradation. (See e.g., Aberle et al., EMBO J. 1997; 16: 3797-804; Latres et al., Oncogene 1999; 18: 849-54; Liu et al., Proc. Natl. Acad. Sci. USA 1999; 96: 6273-8). An alternative degradation pathway has been shown involving ubiquitination induced by complexation with Siah-1 and the C-terminus of APC. (Matsuzawa et al., Mol Cell 2001; 7: 915-926; Liu et al., Mol. Cell 2001; 7: 927-936). In addition to its role as a transcription factor, β-catenin further is involved in cellular adhesion. [Nelson et al., Science 2004; 303: 1483-1487; Ilyas et al., J. Pathol. 1997; 182: 128-137.

β-catenin can be found at the cell surface sites of intercellular contact known as adherens junctions, where it is complexed with E-cadherin. Thus, the breakdown of the E-cadherin-catenin complex can increase cytoplasmic levels of free β-catenin, thereby stimulating transcriptional activity. Thus, activation of the cell surface receptors cRON, epidermal growth factor receptor (EGFR) and c-ErbB2, by liberating β-catenin, can also stimulate canonical Wnt signaling. Other signaling pathways can either activate or facilitate the effects of Wnt signaling. For example, signaling through insulin-like growth factor (IGF) can activate Wnt signaling by “soaking up” available GSK31:3-thereby preventing formation of the “degradation complex.”

As used herein, the term “Wnt agonist” refers to any agent that activates the Wnt/β-catenin pathway, or inhibits the activity and/or expression of inhibitors of Wnt/β-catenin signaling, for example antagonists or inhibitors of GSK-3β activity. A Wnt activating agent as used herein can enhance signaling through the Wnt/β-catenin pathway at any point along the pathway, for example, but not limited to increasing the expression and/or activity of Wnt, or β-catenin or Wnt dependent genes and/or proteins, and decreasing the expression and/or activity of endogenous inhibitors of Wnt and/or β-catenin or decreasing the expression and/or activity of endogenous inhibitors of components of the Wnt/β-catenin pathway, for example decreasing the expression of GSK-3β.

Some non-limiting examples of Wnt pathway agonists include recombinant Wnt proteins (e.g., recombinant Wnt3a), 2-amino-4-[3,4-(methylenedioxy)benzyl-amino]-6-(3-methoxyphenyl)pyrimidine, WAY-316606, GSK-3β inhibitors (e.g., CHIR99021, BIO, SB-216763, those described by Meijer et al. Trends Pharmacol Sci (2004) 25(9):471-480), (2′Z,3′E)-6-Bromoindirubin-3′-oxime, 5-(Furan-2-yl)-N-(3-(1H-imidazol-1-yl)propyl)-1,2-oxazole-3-carboxamide, tankyrase inhibitors (such as e.g., XAV939, WIKI4, JW55, IWR1) and β-catenin activators (e.g., SKL2001, DCA).

### Wnt/β-Catenin Peptides and Polypeptides

Provided herein are peptides and polypeptides (e.g., recombinant polypeptides) useful in the treatment of FSHD. Such peptides and polypeptides act to increase signaling through the Wnt/β-catenin pathway.

A “Wnt protein” is a ligand of the Wnt signaling pathway component which binds to a Frizzled receptor so as to activate Wnt signaling. Specific examples of Wnt proteins include at least 19 members, including proteins translated from the following mRNA sequences: Wnt-1 (RefSeq.: NM_{—}005430), Wnt-2 (RefSeq.: NM_{—}003391), Wnt-2B (also known as Wnt-13) (RefSeq.: NM_{—}004185), Wnt-3 (ReSeq.: NM_{—}030753), Wnt3a (RefSeq.: NM_{—}033131), Wnt-4 (RefSeq.: NM_{—}030761), Wnt-5A (RefSeq.: NM_{—}003392), Wnt-5B (RefSeq.: NM_{—}032642), Wnt-6 (RefSeq.: NM_{—}006522), Wnt-7A (RefSeq.: NM_{—}004625), Wnt-7B (RefSeq.: NM_{—}058238), Wnt-8A (RefSeq.: NM_{—}058244), Wnt-8B (RefSeq.: NM_{—}003393), Wnt-9A (Wnt-14) (RefSeq.: NM_{—}003395), Wnt-9B (Wnt-15) (RefSeq.: NM_{—}003396), Wnt-10A (RefSeq.: NM_{—}025216), Wnt-10B (RefSeq.: NM_{—}003394), Wnt-11 (RefSeq.: NM_{—}004626), Wnt-16 (RefSeq.: NM_{—}016087)). While each member has varying degrees of sequence identity, each contain 23-24 conserved cysteine residues which show highly conserved spacing. McMahon, A P et al, Trends Genet. 8: 236-242 (1992); Miller J R., Genome Biol. 3(1): 3001.1-3001.15 (2002). In one embodiment, the Wnt protein comprises Wnt3a or Wnt9B.

“Polypeptide,” “peptide”, and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer. A polypeptide or amino acid sequence “derived from” a designated polypeptide or protein refers to the origin of the polypeptide. Preferably, the polypeptide or amino acid sequence which is derived from a particular sequence has an amino acid sequence that is essentially identical to that sequence or a portion thereof, wherein the portion consists of at least 10-20 amino acids, preferably at least 20-30 amino acids, more preferably at least 30-50 amino acids, or which is otherwise identifiable to one of ordinary skill in the art as having its origin in the sequence.

Polypeptides derived from another polypeptide may have one or more mutations relative to the starting polypeptide, e.g., one or more amino acid residues which have been substituted with another amino acid residue or which has one or more amino acid residue insertions or deletions. A polypeptide “derived” from another polypeptide will retain therapeutically or physiologically relevant biological activity of the polypeptide from which it is derived. Relevant activity in this context includes, for example, reducing DUX4 expression. By “retain” in such context is meant at least 50% retention, preferably at least 60%, at least 70%, at least 80%, at least 90%, at least 95%, at least 99% or even 100% or greater retention.

A polypeptide can comprise an amino acid sequence which is not naturally occurring. Such variants necessarily have less than 100% sequence identity or similarity with a starting polypeptide molecule. In a preferred embodiment, the variant will have an amino acid sequence from about 75% to less than 100% amino acid sequence identity or similarity with the amino acid sequence of the starting polypeptide, more preferably from about 80% to less than 100%, more preferably from about 85% to less than 100%, more preferably from about 90% to less than 100% (e.g., 91%, 92%, 93%, 94%, 95%, 96%, 97%, 98%, 99%) and most preferably from about 95% to less than 100%, e.g., over the length of the variant molecule. In one embodiment, there is one amino acid difference between a starting polypeptide sequence and the sequence derived therefrom. Identity or similarity with respect to this sequence is defined herein as the percentage of amino acid residues in the candidate sequence that are identical (i.e., same residue) with the starting amino acid residues, after aligning the sequences and introducing gaps, if necessary, to achieve the maximum percent sequence identity. As with polypeptides derived from another, a variant of this kind will retain a therapeutically or physiologically relevant biological activity of the polypeptide from which it is a variant.

In one embodiment, a polypeptide useful in the methods described herein consists of, consists essentially of, or comprises an amino acid sequence encoded by the following mRNA sequences: Wnt-1 (RefSeq.: NM_{—}005430), Wnt-2 (RefSeq.: NM_{—}003391), Wnt-2B (also known as Wnt-13) (RefSeq.: NM_{—}004185), Wnt-3 (ReSeq.: NM_{—}030753), Wnt3a (RefSeq.: NM_{—}033131), Wnt-4 (RefSeq.: NM_{—}030761), Wnt-5A (RefSeq.: NM_{—}003392), Wnt-5B (RefSeq.: NM_{—}032642), Wnt-6 (RefSeq.: NM_{—}006522), Wnt-7A (RefSeq.: NM_{—}004625), Wnt-7B (RefSeq.: NM_{—}058238), Wnt-8A (RefSeq.: NM_{—}058244), Wnt-8B (RefSeq.: NM_{—}003393), Wnt-9A (Wnt-14) (RefSeq.: NM_{—}003395), Wnt-9B (Wnt-15) (RefSeq.: NM_{—}003396), Wnt-10A (RefSeq.: NM_{—}025216), Wnt-10B (RefSeq.: NM_{—}003394), Wnt-11 (RefSeq.: NM_{—}004626), Wnt-16 (RefSeq.: NM_{—}016087)), and functionally active variants thereof. In one embodiment, the Wnt polypeptide is Wnt 3a. In another embodiment, the Wnt polypeptide is Wnt9B. Any of the Wnt polypeptides are contemplated for use in the methods described herein. In the following discussion Wnt3a is referred to—this should be understood to apply equally to other Wnt polypeptides that similarly affect DUX4 expression.

The Wnt3a polypeptides described herein can comprise conservative amino acid substitutions at one or more amino acid residues, e.g., at essential or non-essential amino acid residues but will retain a therapeutically or physiologically relevant activity of a Wnt3a polypeptide as that term is described herein. A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, in a conservative substitution variant, a nonessential amino acid residue in a Wnt3a polypeptide is preferably replaced with another amino acid residue from the same side chain family.

The term “variant” as used herein refers to a polypeptide or nucleic acid that is “substantially similar” to a wild-type Wnt3a polypeptide or nucleic acid. A molecule is said to be “substantially similar” to another molecule if both molecules have substantially similar structures (i.e., they are at least 50% similar in amino acid sequence as determined by BLASTp alignment set at default parameters) and are substantially similar in at least one therapeutically or physiologically relevant function (e.g., effect on DUX4 expression). A variant differs from the naturally occurring polypeptide or nucleic acid by one or more amino acid or nucleic acid deletions, additions, substitutions or side-chain modifications, yet retains one or more therapeutically relevant, specific functions or biological activities of the naturally occurring molecule Amino acid substitutions include alterations in which an amino acid is replaced with a different naturally-occurring or a non-conventional amino acid residue. Some substitutions can be classified as “conservative,” in which case an amino acid residue contained in a polypeptide is replaced with another naturally occurring amino acid of similar character either in relation to polarity, side chain functionality or size. Substitutions encompassed by variants as described herein can also be “non-conservative,” in which an amino acid residue which is present in a peptide is substituted with an amino acid having different properties (e.g., substituting a charged or hydrophobic amino acid with an uncharged or hydrophilic amino acid), or alternatively, in which a naturally-occurring amino acid is substituted with a non-conventional amino acid. Also encompassed within the term “variant,” when used with reference to a polynucleotide or polypeptide, are variations in primary, secondary, or tertiary structure, as compared to a reference polynucleotide or polypeptide, respectively (e.g., as compared to a wild-type polynucleotide or polypeptide). Polynucleotide changes can result in amino acid substitutions, additions, deletions, fusions and truncations in the polypeptide encoded by the reference sequence. Variants can also include insertions, deletions or substitutions of amino acids in the peptide sequence. To be therapeutically useful, such variants will retain a therapeutically or physiologically relevant activity as that term is used herein.

The term “derivative” as used herein refers to peptides which have been chemically modified, for example by ubiquitination, labeling, pegylation (derivatization with polyethylene glycol) or addition of other molecules. A molecule is also a “derivative” of another molecule when it contains additional chemical moieties not normally a part of the molecule. Such moieties can improve the molecule's solubility, absorption, biological half-life, etc. The moieties can alternatively decrease the toxicity of the molecule, or eliminate or attenuate an undesirable side effect of the molecule, etc. Moieties capable of mediating such effects are disclosed in Remington's Pharmaceutical Sciences, 18th edition, A. R. Gennaro, Ed., MackPubl., Easton, Pa. (1990). The term “functional” when used in conjunction with “derivative” or “variant” refers to a polypeptide which possesses a therapeutically or physiologically relevant biological activity that is substantially similar to a biological activity of the entity or molecule of which it is a derivative or variant. By “substantially similar” in this context is meant that at least 50% of the relevant or desired biological activity of a corresponding wild-type peptide is retained. In the instance of reducing DUX4 expression, preferably the variant retains at least 60%, at least 70%, at least 80%, at least 90%, at least 95%, or more, including 100% or even more (i.e., the derivative or variant has improved activity relative to wild-type) of the DUX4-reducing activity of the wild-type.

### Small Molecule Activators of the Wnt/β-Catenin Pathway

As used herein, the term “small molecule” refers to a chemical agent including, but not limited to, peptides, peptidomimetics, amino acids, amino acid analogs, polynucleotides, polynucleotide analogs, aptamers, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Essentially any small molecule activator of the Wnt/β-catenin pathway can be used in the treatment of FSHD using the methods described herein. Screening assays are provided herein for identifying candidate small molecule agents that modulate the Wnt/β-catenin pathway, resulting in a decrease in DUX4 expression and/or activity. It should be noted that some known Wnt/β-catenin pathway inhibitors can act to decrease DUX4 expression in cells having an FSHD phenotype, and thus are contemplated for therapeutic use as described herein.

Some non-limiting examples of Wnt pathway agonists include recombinant Wnt proteins (e.g., recombinant Wnt3a), 2-amino-4-[3,4-(methylenedioxy)benzyl-amino]-6-(3-methoxyphenyl)pyrimidine, GSK-3β inhibitors (e.g., CHIR99021, BIO, SB-216763, and those described by Meijer et al. Trends Pharmacol Sci (2004) 25(9):471-480), (2′Z,3′E)-6-Bromoindirubin-3′-oxime, 5-(Furan-2-yl)-N-(3-(1H-imidazol-1-yl)propyl)-1,2-oxazole-3-carboxamide, tankyrase inhibitors (such as e.g., XAV939, WIKI4, JW55, IWR1) and β-catenin activators (e.g., SKL2001, deoxycholic acid (DCA)).

In one embodiment, the CHIR99021GSK-3β inhibitor has the structure of Formula (I):

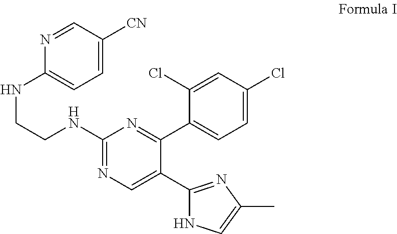 [SMILES: Cc1c[nH]c(-c2cnc(NCCNc3ccc(C#N)cn3)nc2-c2ccc(Cl)cc2Cl)n1]

In another embodiment, the GSK-3β inhibitor BIO has the structure of Formula (II):

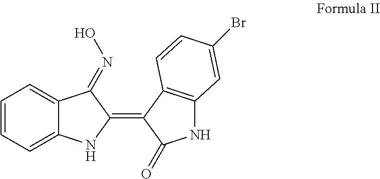 [SMILES: O=C1Nc2cc(Br)ccc2/C1=C1/Nc2ccccc2/C1=N\O]

In another embodiment, the GSK-3β inhibitor SB-216763 has the structure of Formula (III):

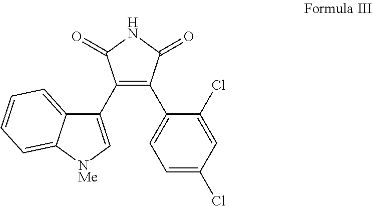 [SMILES: Cn1cc(C2=C(c3ccc(Cl)cc3Cl)C(=O)NC2=O)c2ccccc21]

In another embodiment, the WIKI4 tankyrase inhibitor has the structure of Formula (IV):

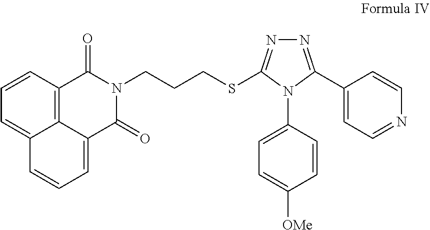 [SMILES: COc1ccc(-n2c(SCCCN3C(=O)c4cccc5cccc(c45)C3=O)nnc2-c2ccncc2)cc1]

In another embodiment, the XAV-939 tankyrase inhibitor has the structure of Formula (V):

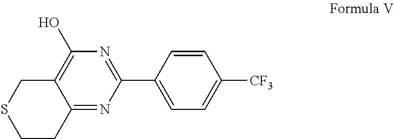 [SMILES: Oc1nc(-c2ccc(C(F)(F)F)cc2)nc2c1CSCC2]

In another embodiment, the IWR tankyrase inhibitor has the structure of Formula (VI):

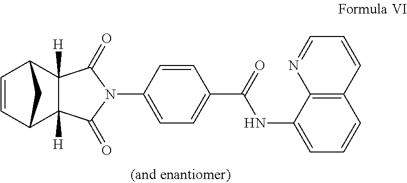 [SMILES: O=C(Nc1cccc2cccnc12)c1ccc(N2C(=O)[C@@H]3[C@H](C2=O)[C@@H]2C=C[C@H]3C2)cc1]

In another embodiment, the JW55 tankyrase inhibitor has the structure of Formula (VII):

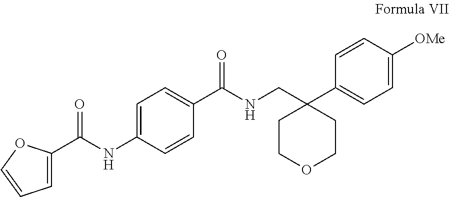 [SMILES: COc1ccc(C2(CNC(=O)c3ccc(NC(=O)c4ccco4)cc3)CCOCC2)cc1]

In another embodiment, the β-catenin activator SKL2001 has the structure of Formula (VIII):

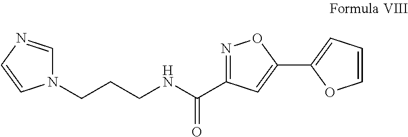 [SMILES: O=C(NCCCn1ccnc1)c1cc(-c2ccco2)on1]

In another embodiment, the β-catenin activator DCA has the structure of Formula (IX):

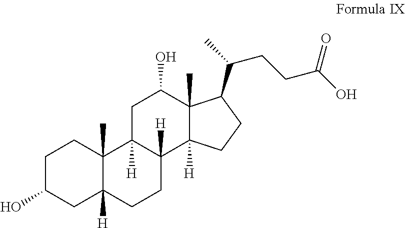

### Nucleic Acid Inhibitors of GSK-3β

A powerful approach for inhibiting the expression of selected target polypeptides, such as GSK3-β, is through the use of RNA interference agents. “RNA interference (RNAi)” is an evolutionally conserved process whereby the expression or introduction of RNA of a sequence that is identical or highly similar to a target gene results in the sequence specific degradation or specific post-transcriptional gene silencing (PTGS) of messenger RNA (mRNA) transcribed from that targeted gene (see Coburn, G. and Cullen, B. (2002) J. of Virology 76(18):9225), thereby inhibiting expression of the target gene. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex (termed “RNA induced silencing complex,” or “RISC”) that recognizes and cleaves target mRNAs. RNAi can also be initiated by introducing nucleic acid molecules, e.g., synthetic siRNAs or RNA interfering agents, to inhibit or silence the expression of target genes. As used herein, “inhibition of target gene expression” includes any decrease in expression or protein activity or level of the target gene or protein encoded by the target gene as compared to a situation wherein no RNA interference has been induced. The decrease will be of at least 10%, 20%, 30%, 40%, 50%, 60%, 70%, 80%, 90%, 95% or 99% or more as compared to the expression of a target gene or the activity or level of the protein encoded by a target gene which has not been targeted by an RNA interfering agent.

The terms “RNA interference agent” and “RNA interference” as they are used herein are intended to encompass those forms of gene silencing mediated by double-stranded RNA, regardless of whether the RNA interfering agent comprises an siRNA, miRNA, shRNA or other double-stranded RNA molecule. “Short interfering RNA” (siRNA), also referred to herein as “small interfering RNA” is defined as an RNA agent which functions to inhibit expression of a target gene, e.g., by RNAi. An siRNA can be chemically synthesized, can be produced by in vitro transcription, or can be produced within a host cell. In one embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, 22, or 23 nucleotides in length, and can contain a 3′ and/or 5′ overhang on each strand having a length of about 0, 1, 2, 3, 4, or 5 nucleotides. The length of the overhang is independent between the two strands, i.e., the length of the overhang on one strand is not dependent on the length of the overhang on the second strand. Preferably the siRNA is capable of promoting RNA interference through degradation or specific post-transcriptional gene silencing (PTGS) of the target messenger RNA (mRNA).

Methods for designing an siRNA or other RNA interference agent for inhibiting e.g., GSK3-β, which in turn activates the Wnt/β-catenin pathway as described herein are known to those of skill in the art.

In one embodiment, the RNA interference agent is delivered or administered to a subject in a pharmaceutically acceptable carrier. Additional carrier agents, such as liposomes, can be added to the pharmaceutically acceptable carrier. In another embodiment, the RNA interference agent is delivered by a vector encoding small hairpin RNA (shRNA) in a pharmaceutically acceptable carrier to the cells in an organ of an individual. The shRNA is converted by the cells after transcription into siRNA capable of targeting, for example, DUX4 or GSK3-β.

In another embodiment, a nucleic acid sequence encoding the RNA interference agent is administered to the subject or cell (e.g., a plasmid or viral vector, e.g., a lentiviral vector. Such vectors can be used as described, for example, in Xiao-Feng Qin et al. Proc. Natl. Acad. Sci. U.S.A., 100: 183-188). In such an embodiment, the nucleic acid sequence can comprise an expression vector. In one embodiment, the vector is a regulatable vector, such as a tetracycline inducible vector. Methods described, for example, in Wang et al. Proc. Natl. Acad. Sci. 100: 5103-5106, using pTet-On vectors (BD Biosciences Clontech, Palo Alto, Calif.) can be used. In one embodiment, the RNA interference agents used in the methods described herein are taken up actively by cells in vivo following intravenous injection, e.g., hydrodynamic injection, without the use of a vector, illustrating efficient in vivo delivery of the RNA interfering agents. One method to deliver the siRNAs is by topical administration in an appropriate pharmaceutically acceptable carrier. Other delivery methods include delivery of the RNA interfering agents, e.g., the siRNAs or shRNAs, using a basic peptide by conjugating or mixing the RNA interfering agent with a basic peptide, e.g., a fragment of a TAT peptide, mixing with cationic lipids or formulating into particles. The RNA interference agents, e.g., the siRNAs targeting GSK3-β mRNA, can be delivered singly, or in combination with other RNA interference agents, e.g., siRNAs, such as, for example siRNAs directed to other cellular genes. siRNAs can also be administered in combination with other pharmaceutical agents which are used to treat or prevent diseases or disorders comprising FSHD.

Synthetic siRNA molecules, including shRNA molecules, can be obtained using a number of techniques known to those of skill in the art. For example, the siRNA molecule can be chemically synthesized or recombinantly produced using methods known in the art, such as using appropriately protected ribonucleoside phosphoramidites and a conventional DNA/RNA synthesizer (see, e.g., Elbashir, S. M. et al. (2001) Nature 411:494-498; Elbashir, S. M., W. Lendeckel and T. Tuschl (2001) Genes & Development 15:188-200; Harborth, J. et al. (2001) J. Cell Science 114:4557-4565; Masters, J. R. et al. (2001) Proc. Natl. Acad. Sci., USA 98:8012-8017; and Tuschl, T. et al. (1999) Genes & Development 13:3191-3197). Alternatively, several commercial RNA synthesis suppliers are available including, but not limited to, Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK). As such, siRNA molecules are not overly difficult to synthesize and are readily provided in a quality suitable for RNAi. In addition, dsRNAs can be expressed as stem loop structures encoded by plasmid vectors, retroviruses and lentiviruses (Paddison, P. J. et al. (2002) Genes Dev. 16:948-958; McManus, M. T. et al. (2002) RNA 8:842-850; Paul, C. P. et al. (2002) Nat. Biotechnol. 20:505-508; Miyagishi, M. et al. (2002) Nat. Biotechnol. 20:497-500; Sui, G. et al. (2002) Proc. Natl. Acad. Sci., USA 99:5515-5520; Brummelkamp, T. et al. (2002) Cancer Cell 2:243; Lee, N. S., et al. (2002) Nat. Biotechnol. 20:500-505; Yu, J. Y., et al. (2002) Proc. Natl. Acad. Sci., USA 99:6047-6052; Zeng, Y., et al. (2002) Mol. Cell 9:1327-1333; Rubinson, D. A., et al. (2003) Nat. Genet. 33:401-406; Stewart, S. A., et al. (2003) RNA 9:493-501). These vectors generally have a polIII promoter upstream of the dsRNA and can express sense and antisense RNA strands separately and/or as a hairpin structure.

### Delivery of RNA Interfering Agents

Methods of delivering RNA interference agents, e.g., an siRNA, or vectors containing an RNA interference agent, to the target cells, e.g., muscle cell, or other desired target cells, for uptake include injection of a composition containing the RNA interference agent, e.g., an siRNA, or directly contacting the cell, e.g., a muscle cell, with a composition comprising an RNA interference agent, e.g., an siRNA. For example, the RNA interference agent can be delivered directly to the muscle or the blood vessel supplying the muscle. In another embodiment, the RNA interference agent, e.g., an siRNA can be injected directly into any blood vessel, such as vein, artery, venule or arteriole, via, e.g., hydrodynamic injection or catheterization. Administration can be by a single injection or by two or more injections. The RNA interference agent is delivered in a pharmaceutically acceptable carrier. One or more RNA interference agents can be used simultaneously. In one embodiment, a single siRNA that targets human GSK-3β is used. In another embodiment, one or more siRNAs that target human GSK-3β is used. In one embodiment, specific cells are targeted with RNA interference, limiting potential side effects of RNA interference caused by non-specific targeting of RNA interference. The method can use, for example, a complex or a fusion molecule comprising a cell targeting moiety and an RNA interference binding moiety that is used to deliver RNA interference effectively into cells. The siRNA or RNA interference-inducing molecule binding moiety is a protein or a nucleic acid binding domain or fragment of a protein, and the binding moiety is fused to a portion of the targeting moiety. The location of the targeting moiety can be either in the carboxyl-terminal or amino-terminal end of the construct or in the middle of the fusion protein. Viral-mediated delivery of siRNAs to cells in vitro and in vivo is known in the art (see e.g., Manjunath et al. (2010) Discovery Med 9(48):418-30; Castanotto et al. (2009) Nature 457:426-433). Plasmid- or viral-mediated delivery mechanisms of shRNA can also be employed to deliver shRNAs to cells in vitro and in vivo as reviewed by Manjunath et al. (2010) Discovery Med 9(48):418-30; Castanotto et al. (2009) Nature 457:426-433; Aigner et al. (2008) Curr Pharm Des 14:3603-3619; Whitehead et al. (2009) Nat Rev Drug Discov 8:129-138; Laufer et al. (2010) RNA Technologies and Their Applications, Chapter title “Selected Strategies for the Delivery of siRNA In Vitro and In Vivo” Erdmann and Barciszewski (eds)). The RNA interference agents, e.g., the siRNAs or shRNAs, can be introduced along with components that perform one or more of the following activities: enhance uptake of the RNA interfering agents, e.g., siRNA, by the cell, e.g., lymphocytes or other cells, inhibit annealing of single strands, stabilize single strands, or otherwise facilitate delivery to the target cell and increase inhibition of the target gene, e.g., GSK3-β. The dose of the particular RNA interfering agent will be in an amount necessary to effect RNA interference, e.g., post translational gene silencing (PTGS), of the particular target gene, thereby leading to inhibition of target gene expression or inhibition of activity or level of the protein encoded by the target gene.

### Pharmaceutical Compositions

Provided herein are compositions that are useful for treating and preventing FSHD in a subject. In one embodiment, the composition is a pharmaceutical composition. The composition can comprise a therapeutically or prophylactically effective amount of a Wnt peptide or polypeptide (e.g., native or recombinant), a polynucleotide encoding a member of the Wnt/β-catenin pathway (e.g., Wnt3a), or a recombinant virus expressing Wnt. In another embodiment, the composition can comprise a therapeutically or prophylactically effective amount of a GSK-3β inhibitor, an activator of DNMT-2 or an activator of SMCHD1.

The composition can optionally include a carrier, such as a pharmaceutically acceptable carrier. Pharmaceutically acceptable carriers are determined in part by the particular composition being administered, as well as by the particular method used to administer the composition. Accordingly, there is a wide variety of suitable formulations of pharmaceutical compositions. Formulations suitable for parenteral administration can be formulated, for example, for intravenous, intramuscular, intradermal, intraperitoneal, and subcutaneous routes. Carriers can include aqueous isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, preservatives, liposomes, microspheres and emulsions.

Therapeutic compositions contain a physiologically tolerable carrier together with an active agent as described herein, dissolved or dispersed therein as an active ingredient. As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. A pharmaceutically acceptable carrier will not promote the raising of an immune response to an agent with which it is admixed, unless so desired. The preparation of a pharmaceutical composition that contains active ingredients dissolved or dispersed therein is well understood in the art and need not be limited based on formulation. Typically such compositions are prepared as injectable either as liquid solutions or suspensions; however, solid forms suitable for solution, or suspensions in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients include, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. The therapeutic composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Examples of such additional liquid phases are glycerin, vegetable oils such as cottonseed oil, and water-oil emulsions. The amount of an active agent used in the methods described herein that will be effective in the treatment of a particular disorder or condition will depend on the nature of the disorder or condition, and can be determined by standard clinical techniques.

A pharmaceutical composition can contain DNA encoding one or more of the members of the Wnt/β-catenin pathway, such that the polypeptide is generated in situ. As noted above, the DNA can be present within any of a variety of delivery systems known to those of ordinary skill in the art, including nucleic acid expression systems, bacterial and viral expression systems. Numerous gene delivery techniques are well known in the art, such as those described by Rolland, 1998, Crit. Rev. Therap. Drug Carrier Systems 15:143-198, and references cited therein. Appropriate nucleic acid expression systems contain the necessary DNA sequences for expression in the patient (such as a suitable promoter and terminating signal. In a preferred embodiment, the DNA can be introduced using a viral expression system (e.g., vaccinia or other pox virus, retrovirus, or adenovirus), which can involve the use of a non-pathogenic (defective), replication competent virus. Suitable systems are reviewed, for example, in Siddhesh et al. (2005) AAPS J 7(1):E61-E77. Techniques for incorporating DNA into such expression systems are well known to those of ordinary skill in the art. The DNA can also be “naked,” as reviewed, for example, in Siddhesh et al. (2005) AAPS J 7(1):E61-E77. The uptake of naked DNA can be increased by coating the DNA onto biodegradable beads, which are efficiently transported into the cells.

While any suitable carrier known to those of ordinary skill in the art can be employed in the pharmaceutical compositions of this invention, the type of carrier will vary depending on the mode of administration. Compositions of the present invention can be formulated for any appropriate manner of administration, including for example, topical, oral, nasal, intravenous, intracranial, intraperitoneal, subcutaneous or intramuscular administration. For parenteral administration, such as intramuscular or subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactate polyglycolate) can also be employed as carriers for the pharmaceutical compositions. Suitable biodegradable microspheres are disclosed, for example, in U.S. Pat. Nos. 4,897,268 and 5,075,109. Such compositions can also comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, chelating agents such as EDTA or glutathione, adjuvants (e.g., aluminum hydroxide) and/or preservatives. Alternatively, compositions as described herein can be formulated as a lyophilizate. Compounds can also be encapsulated within liposomes using well known technology. The compositions described herein can be administered as part of a sustained release formulation (i.e., a formulation such as a capsule or sponge that affects a slow release of compound following administration). Such formulations can generally be prepared using well known technology and administered by, for example, oral, rectal or subcutaneous implantation, or by implantation at the desired target site. Sustained-release formulations can contain a polypeptide, polynucleotide dispersed in a carrier matrix and/or contained within a reservoir surrounded by a rate controlling membrane. Carriers for use within such formulations are biocompatible, and can also be biodegradable; preferably the formulation provides a relatively constant level of active component release. The amount of active compound contained within a sustained release formulation depends upon the site of implantation, the rate and expected duration of release and the nature of the condition to be treated or prevented.

### Expression Vectors

A nucleic acid encoding, for example, a Wnt3a polypeptide can be expressed in a cell (e.g., a muscle cell) using an expression vector. The term “vector” refers to a carrier DNA molecule into which a nucleic acid sequence can be inserted for introduction into a host cell. An “expression vector” is a specialized vector that contains the necessary regulatory regions needed for expression of a gene of interest in a host cell. In some embodiments the gene of interest is operably linked to another sequence in the vector. In some embodiments, it is preferred that the viral vectors are replication defective, which can be achieved for example by removing viral nucleic acids that encode for replication. A replication defective viral vector will still retain its infective properties and enters the cells in a similar manner as a replicating vector, however once admitted to the cell a replication defective viral vector does not reproduce or multiply.

Many viral vectors or virus-associated vectors are known in the art. Such vectors can be used as carriers of a nucleic acid construct into the cell. Constructs can be integrated and packaged into non-replicating, defective viral genomes like Adenovirus, Adeno-associated virus (AAV), or Herpes simplex virus (HSV) or others, including retroviral and lentiviral vectors, for infection or transduction into cells. The vector can be incorporated into the cell's genome. The constructs can include viral sequences for transfection, if desired. Alternatively, the construct can be incorporated into vectors capable of episomal replication, e.g., EPV and EBV vectors. The inserted material of the vectors described herein can be operatively linked to an expression control sequence wherein the expression control sequence controls and regulates the transcription and translation of that polynucleotide sequence. The term “operatively linked” can include having an appropriate start signal (e.g., ATG) in front of the polynucleotide sequence to be expressed, and maintaining the correct reading frame to permit expression of the polynucleotide sequence under the control of the expression control sequence, and production of the desired polypeptide encoded by the polynucleotide sequence. In some examples, transcription of an inserted material is under the control of a promoter sequence (or other transcriptional regulatory sequence) which controls the expression of the recombinant gene in a cell-type in which expression is intended. It will also be understood that the inserted material can be under the control of transcriptional regulatory sequences which are the same or which are different from those sequences which control transcription of the naturally-occurring form of a protein. In some instances the promoter sequence is recognized by the synthetic machinery of the cell, or introduced synthetic machinery, required for initiating transcription of a specific gene.

An “inducible promoter” is a promoter that is capable of directly or indirectly activating transcription of one or more DNA sequences or genes in response to a “regulatory agent” (e.g., doxycycline), or a “stimulus” (e.g., heat). In the absence of a “regulatory agent” or “stimulus”, the DNA sequences or genes will not be substantially transcribed. The term “not substantially transcribed” or “not substantially expressed” means that the level of transcription is at least 100-fold lower than the level of transcription observed in the presence of an appropriate stimulus or regulatory agent; preferably at least 200-fold, 300-fold, 400-fold, 500-fold or more. As used herein, the terms “stimulus” and/or “regulatory agent” refers to a chemical agent, such as a metabolite, a small molecule, or a physiological stress directly imposed upon the organism such as cold, heat, toxins, or through the action of a pathogen or disease agent. A recombinant cell containing an inducible promoter can be exposed to a regulatory agent or stimulus by externally applying the agent or stimulus to the cell or organism by exposure to the appropriate environmental condition or the operative pathogen. Inducible promoters initiate transcription only in the presence of a regulatory agent or stimulus. Examples of inducible promoters include the tetracycline response element and promoters derived from the β-interferon gene, heat shock gene, metallothionein gene or any obtainable from steroid hormone-responsive genes. Inducible promoters which can be used in performing the methods as described herein include those regulated by hormones and hormone analogs such as progesterone, ecdysone and glucocorticoids as well as promoters which are regulated by tetracycline, heat shock, heavy metal ions, interferon, and lactose operon activating compounds. Tissue specific expression has been well characterized in the field of gene expression and tissue specific and inducible promoters are well known in the art. These promoters are used to regulate the expression of the foreign gene after it has been introduced into the target cell.

The promoter sequence can be a “muscle-specific promoter,” which means a nucleic acid sequence that serves as a promoter, i.e., regulates expression of a selected nucleic acid sequence operably linked to the promoter, and which affects expression of the selected nucleic acid sequence in specific cells, preferably in skeletal muscle cells. The term also covers so-called “leaky” promoters, which regulate expression of a selected nucleic acid primarily in one tissue, but cause expression in other tissues as well. For expression of an exogenous gene specifically in muscle cells, a muscle-specific promoter, such as a desmin, α-actin, or the Mb promoter, can be used. Other cell specific promoters active in mammalian cells are also contemplated herein. Such promoters provide a convenient means for controlling expression of the exogenous gene in a cell of a cell culture or within a mammal.

In some embodiments, the expression vector is a lentiviral vector. Lentiviral vectors useful for the methods and compositions described herein can comprise a eukaryotic promoter. The promoter can be any inducible promoter, including synthetic promoters that can function as a promoter in a eukaryotic cell. For example, the eukaryotic promoter can be, but is not limited to, ecdysone inducible promoters, E1a inducible promoters, tetracycline inducible promoters etc., as are well known in the art. In addition, the lentiviral vectors used herein can further comprise a selectable marker, which can comprise a promoter and a coding sequence for a selectable trait. Nucleotide sequences encoding selectable markers are well known in the art, and include those that encode gene products conferring resistance to antibiotics or anti-metabolites, or that supply an auxotrophic requirement. Examples of such sequences include, but are not limited to, those that encode thymidine kinase activity, or resistance to methotrexate, ampicillin, kanamycin, chloramphenicol, or zeocin, among many others.

### Delivery of Nucleic Acids Encoding a Wnt/β-Catenin Activator

The principles of gene delivery, as well as disclosures of exemplary methods and uses for gene therapy, are reviewed by e.g., Siddhesh et al. (2005) AAPS J 7(1):E61-E77, Delalande et al. (2013) Gene 525(2):191-9; Want et al. (2013) Curr Pharm Biotechnol 14(1):46-60; Schlenk et al. (2013) Ther Deliv 4(1):95-113; Gan et al. (2013) Front Biosci 5:188-203; Nimesh (2012) Ther Deliv 3(11):1347-56; Bersenev et al. (2012) Regen Med 7(6 Suppl):50-6; Podolska et al. (2012) Adv Clin Exp Med 21(4):525-34; Gupta et al. (2012) Crit Rev Ther Drug Carrier Syst 29(6):447-85; Mellott et al. (2013) Ann Biomed Eng 41(3):446-68; Emeagi et al. (2013) 13(4):602-25; and Samal et al. (2012) Chem Soc Rev 41(21):7147-94, all of which are incorporated herein by reference in their entirety.

In one embodiment, the nucleic acid encoding e.g., Wnt3a can be administered to a patient by any one of several gene therapy techniques known to those of skill in the art. In general, gene therapy can be accomplished by either direct transformation of target cells within the mammalian subject (in vivo gene therapy) or transformation of cells in vitro and subsequent implantation of the transformed cells into the mammalian subject (ex vivo gene therapy).

In one embodiment of the methods described herein, DNA encoding a Wnt3a polypeptide can be introduced into the somatic cells of an animal (particularly mammals, including humans) in order to provide a treatment of a disease or condition that responds to the composition. Most preferably, viral or retroviral vectors are employed for this purpose.

Retroviral vectors are a common mode of delivery and in this context are often retroviruses from which viral genes have been removed or altered so that viral replication does not occur in cells infected with the vector. Viral replication functions are provided by the use of retrovirus “packaging” cells that produce the viral proteins required for nucleic acid packaging but that do not produce infectious virus.

Introduction of the retroviral vector DNA into packaging cells results in production of virions that carry vector RNA and can infect target cells, but such that no further virus spread occurs after infection. To distinguish this process from a natural virus infection where the virus continues to replicate and spread, the term transduction rather than infection is often used.

In one embodiment, the methods use a recombinant lentivirus for the delivery and expression of e.g., a Wnt3a polypeptide or peptide in either dividing or non-dividing mammalian cells. The HIV-1 based lentivirus can effectively transduce a broader host range than the Moloney Leukemia Virus (MoMLV)-based retroviral systems. Preparation of the recombinant lentivirus can be achieved using the pLenti4/V5-DEST™, pLenti6/V5-DEST™ or pLenti vectors together with ViraPower™ Lentiviral Expression systems from INVITROGEN.

Examples of use of lentiviral vectors for gene therapy are described in the following references and are hereby incorporated by reference in their entirety (Emeagi et al. (2013) 13(4):602-25; Klein, C. and Baum, C. (2004). Hematol. J., 5, 103-111; Morizono, K. et. al. (2005). Nat. Med., 11, 346-352; Di Domenico, C. et. al. (2005), Hum. Gene Ther., 16, 81-90; Kim, E. Y., Hong, Y. B., Lai, Z., Kim, H. J., Cho, Y.-H., Brady, R. O. and Jung, S.-C. (2004). Biochem. Biophys. Res. Comm, 318, 381-390).

Non-retroviral vectors also have been used in genetic therapy. One such alternative is the adenovirus (Gan et al. (2013) Front Biosci 5:188-203; Podolska et al. (2012) Adv Clin Exp Med 21(4):525-34; Cupelli et al. (2011) Curr Opin Virol 2-84:91). Major advantages of adenovirus vectors are their potential to carry large segments of DNA (36 Kb genome), a very high titer (10¹¹ particles/ml), ability to infect non-replicating cells, and suitability for infecting tissues in situ. Similarly, herpes viruses can also prove valuable for human gene therapy (Cuchet et al. (2007) Expert Opin Biol Ther 7(7):975-95). Of course, any other suitable viral vector can be used for the genetic therapy for the delivery of a nucleic acid encoding Wnt3a as described herein.

The virion used for gene therapy can be any virion known in the art including, but not limited to, those derived from adenovirus, adeno-associated virus (AAV), retrovirus, and lentivirus. Recombinant viruses provide a versatile system for gene expression studies and therapeutic applications.

In one embodiment, the methods described herein use a recombinant adeno-associated virus (rAAV) vector for the expression of e.g., a Wnt3a polypeptide or peptide, or e.g., a fusion protein including a peptide as described herein. Using rAAV vectors, genes can be delivered into a wide range of host cells including many different human and non-human cell lines or tissues. Because AAV is non-pathogenic and does not elicit an immune response, a multitude of pre-clinical studies have reported excellent safety profiles. rAAVs are capable of transducing a broad range of cell types and transduction is not dependent on active host cell division. High titers, >10⁸ viral particle/ml, are easily obtained in the supernatant and 10¹¹-10¹² viral particle/ml can be obtained with further concentration. The transgene is integrated into the host genome, so expression is long term and stable.

Although local administration will most likely be preferred, a nucleic acid encoding a Wnt polypeptide, e.g., Wnt3a used in the methods described herein can be delivered systemically via in vivo gene therapy. Systemic treatment involves transfecting target cells with the DNA of interest, e.g., DNA encoding a Wnt3a polypeptide or peptide, expressing the coded peptide/protein in that cell, and the capability of the transformed cell to subsequently secrete the manufactured peptide/protein into the blood.

Also contemplated herein are a variety of methods to accomplish in vivo transformation including mechanical means (e.g., direct injection of nucleic acid into target cells or particle bombardment), recombinant viruses, liposomes, and receptor-mediated endocytosis (RME). Such methods are known to those of skill in the art and are not described in detail herein.

Another gene transfer method for use in humans is the transfer of plasmid DNA in liposomes directly to human cells in situ (see e.g., Kaneda et al. (2011) Curr Gene Ther 11(6):434-41). Plasmid DNA may be easy to certify for use in human gene therapy because, unlike retroviral vectors, it can be purified to homogeneity. In addition to liposome-mediated DNA transfer, several other physical DNA transfer methods, such as those targeting the DNA to receptors on cells by conjugating the plasmid DNA to proteins, are contemplated for use with the methods described herein (see e.g., Zhao et al. (2012) Expert Opin Drug Deliv 9(1):127-39).

### Dosage and Administration

Treatment includes prophylaxis and therapy. Prophylaxis or treatment can be accomplished by a single direct injection at a single time point or multiple time points. Administration can also be nearly simultaneous to multiple sites. Patients or subjects include mammals, such as human, bovine, equine, canine, feline, porcine, and ovine animals as well as other veterinary subjects. Preferably, the patients or subjects are human.

In one aspect, the methods described herein provide a method for treating FSHD in a subject. In one embodiment, the subject can be a mammal. In another embodiment, the mammal can be a human, although the approach is effective with respect to all mammals. The method comprises administering to the subject an effective amount of a pharmaceutical composition comprising an activator of the Wnt/β-catenin pathway, a GSK-3β inhibitor, an activator of SCMHD1, or an activator of DNMT-1 in a pharmaceutically acceptable carrier. In some embodiments, the method comprises administering to the subject an effective amount of a pharmaceutical composition comprising an inhibitor of GSK-3β, for example, a binding protein, such as an antibody or a peptide. In other embodiments, the inhibitor of GSK-3β comprises a small molecule or an RNA interference molecule (e.g., siRNA, shRNA etc.).

The dosage range for the agent depends upon the potency, and includes amounts large enough to produce the desired effect, e.g., reduction in at least one symptom of FSHD. The dosage should not be so large as to cause unacceptable adverse side effects. Generally, the dosage will vary with the type of inhibitor (e.g., an antibody or fragment, small molecule, siRNA, etc.) or activator (e.g., recombinant polypeptide, peptide, peptidomimetic, small molecule, etc.), and with the age, condition, and sex of the patient. The dosage can be determined by one of skill in the art and can also be adjusted by the individual physician in the event of any complication. Typically, the dosage ranges from 0.001 mg/kg body weight to 5 g/kg body weight. In some embodiments, the dosage range is from 0.001 mg/kg body weight to 1 g/kg body weight, from 0.001 mg/kg body weight to 0.5 g/kg body weight, from 0.001 mg/kg body weight to 0.1 g/kg body weight, from 0.001 mg/kg body weight to 50 mg/kg body weight, from 0.001 mg/kg body weight to 25 mg/kg body weight, from 0.001 mg/kg body weight to 10 mg/kg body weight, from 0.001 mg/kg body weight to 5 mg/kg body weight, from 0.001 mg/kg body weight to 1 mg/kg body weight, from 0.001 mg/kg body weight to 0.1 mg/kg body weight, from 0.001 mg/kg body weight to 0.005 mg/kg body weight. Alternatively, in some embodiments the dosage range is from 0.1 g/kg body weight to 5 g/kg body weight, from 0.5 g/kg body weight to 5 g/kg body weight, from 1 g/kg body weight to 5 g/kg body weight, from 1.5 g/kg body weight to 5 g/kg body weight, from 2 g/kg body weight to 5 g/kg body weight, from 2.5 g/kg body weight to 5 g/kg body weight, from 3 g/kg body weight to 5 g/kg body weight, from 3.5 g/kg body weight to 5 g/kg body weight, from 4 g/kg body weight to 5 g/kg body weight, from 4.5 g/kg body weight to 5 g/kg body weight, from 4.8 g/kg body weight to 5 g/kg body weight. In one embodiment, the dose range is from 5 μg/kg body weight to 30μ/kg body weight. Alternatively, the dose range will be titrated to maintain serum levels between 5 μg/mL and 30 μg/mL.

Administration of the doses recited above can be repeated for a limited period of time. In some embodiments, the doses are given once a day, or multiple times a day, for example but not limited to three times a day. In another embodiment, the doses recited above are administered daily for several weeks or months. The duration of treatment depends upon the subject's clinical progress and responsiveness to therapy. Where FSHD is a chronic, genetically based disease, long-term therapy is specifically contemplated to keep DUX4 levels in check. Continuous, relatively low maintenance doses are contemplated after an initial higher therapeutic dose.

A therapeutically effective amount is an amount of an agent that is sufficient to produce a statistically significant, measurable change in at least one symptom of FSHD (see “Efficacy Measurement” below). Alternatively, a therapeutically effective amount is an amount of an agent that is sufficient to produce a statistically significant, measurable change in the expression level of DUX4 (e.g., a decrease in DUX4 expression) in the subject. Such effective amounts can be gauged in clinical trials as well as animal studies for a given agent.

Agents useful in the methods and compositions described herein can be administered directly to the muscle (e.g., intramuscular injection) or can be administered orally. It is also contemplated herein that the agents can also be delivered intravenously (by bolus or continuous infusion), by inhalation, intranasally, intraperitoneally, intramuscularly, subcutaneously, intracavity, and can be delivered by peristaltic means, if desired, or by other means known by those skilled in the art. In one embodiment it is preferred that the agents for the methods described herein are administered directly to a muscle or muscle group (e.g., during surgery or by direct injection). The agent can be administered systemically, if so desired.

Preferably, the pharmaceutically acceptable formulation used to administer the active compound provides sustained delivery, such as “slow release” of the active compound to a subject. For example, the formulation can deliver the agent or composition for at least one, two, three, or four weeks after the pharmaceutically acceptable formulation is administered to the subject. Preferably, a subject to be treated in accordance with the methods described herein is treated with the active composition for at least 30 days (either by repeated administration or by use of a sustained delivery system, or both).

As used herein, the term “sustained delivery” is intended to include continual delivery of the composition in vivo over a period of time following administration, preferably at least several days, a week, several weeks, one month or longer. Sustained delivery of the active compound can be demonstrated by, for example, the continued therapeutic effect of the composition over time (such as sustained delivery of the agents can be demonstrated by continued improvement or maintained improvement in FSHD symptoms in a subject).

Preferred approaches for sustained delivery include use of a polymeric capsule, a minipump to deliver the formulation, a biodegradable implant, or implanted transgenic autologous cells (as described in U.S. Pat. No. 6,214,622). Implantable infusion pump systems (such as Infusaid; see such as Zierski, J. et al, 1988; Kanoff, R. B., 1994) and osmotic pumps (sold by ALZA CORPORATION) are available in the art. Another mode of administration is via an implantable, externally programmable infusion pump. Suitable infusion pump systems and reservoir systems are described in U.S. Pat. No. 5,368,562 by Blomquist and U.S. Pat. No. 4,731,058 by Doan, developed by Pharmacia Deltec Inc.

Therapeutic compositions containing at least one agent can be conventionally administered in a unit dose. The term “unit dose” when used in reference to a therapeutic composition refers to physically discrete units suitable as unitary dosage for the subject, each unit containing a predetermined quantity of active material calculated to produce the desired therapeutic effect in association with the required physiologically acceptable diluent, i.e., carrier, or vehicle.

The compositions are administered in a manner compatible with the dosage formulation, and in a therapeutically effective amount. The quantity to be administered and timing depends on the subject to be treated, capacity of the subject's system to utilize the active ingredient, and degree of therapeutic effect desired. An agent can be targeted by means of a targeting moiety, such as e.g., an antibody or targeted liposome technology. In some embodiments, an agent can be targeted to a tissue by using bispecific antibodies, for example produced by chemical linkage of an anti-ligand antibody (Ab) and an Ab directed toward a specific target. To avoid the limitations of chemical conjugates, molecular conjugates of antibodies can be used for production of recombinant bispecific single-chain Abs directing ligands and/or chimeric inhibitors at cell surface molecules. The addition of an antibody to an agent permits the agent to accumulate additively at the desired target site (e.g., muscle). Antibody-based or non-antibody-based targeting moieties can be employed to deliver a ligand or the inhibitor to a target site. Preferably, a natural binding agent for an unregulated or disease associated antigen is used for this purpose.

Precise amounts of active ingredient required to be administered depend on the judgment of the practitioner and are particular to each individual. However, suitable dosage ranges for systemic application are disclosed herein and depend on the route of administration. Suitable regimes for administration are also variable, but are typified by an initial administration followed by repeated doses at one or more intervals by a subsequent injection or other administration. Alternatively, continuous intravenous infusion sufficient to maintain concentrations in the blood or skeletal muscle tissue in the ranges specified for in vivo therapies are contemplated.

### Efficacy Measurement

The efficacy of a given treatment for FSHD can be determined by the skilled clinician. However, a treatment is considered “effective treatment,” as the term is used herein, if any one or all of the signs or symptoms of FSHD is/are altered in a beneficial manner (e.g., improved strength, reduced muscle atrophy etc.), other clinically accepted symptoms or markers of disease are improved, or even ameliorated, e.g., by at least 10% following treatment with an agent comprising an activator of the Wnt/β-catenin pathway, a GSK-3β inhibitor, an activator of DNMT-1 or an activator of SMCHD1. Efficacy can also be measured by failure of an individual to worsen as assessed by stabilization of FSHD, hospitalization or need for medical interventions (i.e., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing progression of FSHD; or (2) relieving the disease, e.g., causing regression of symptoms; and (3) preventing or reducing the likelihood of the development of FSHD or preventing secondary issues associated with the FSHD (e.g., injury, accidents, etc.).

An effective amount for the treatment of a disease means that amount which, when administered to a mammal in need thereof, is sufficient to result in effective treatment as that term is defined herein, for that disease. Efficacy of an agent can be determined by assessing physical indicators of FSHD, such as e.g., improved muscle strength, reduced atrophy etc.

Clinically, an effective dose of an agent that increases signaling through the Wnt/β-catenin pathway as described herein, or effective regimen, is a combination of dose and dosing that provides for an improvement in the symptoms associated with FSHD.

Clinical tests for assessing muscular dystrophy and FSHD are known to those of skill in the art and can be used in the clinical setting by those of skill in the art of medicine. The treatment of FSHD can be monitored by an electromyogram, which measures the muscle's response to a stimulation of its nerve supply (nerve conduction study) and the electrical activity in the muscle (needle electrode examination). This test can be used to assess the presence of muscle weakness independent of the nervous system and can also indicate the degree of muscular weakness before and after treatment as described herein. Alternatively, enzymes released from damaged muscle (e.g., creatine kinase) can be measured in a blood sample obtained from a patient in the presence and absence of treatment as described herein. In the absence of muscle trauma, such enzymes can be used to assess the degree of muscular dystrophy and/or the efficacy of a particular treatment of FSHD.

### Screening Assays

Screening assays as contemplated herein can be used to identify modulators, i.e., candidate or test compounds or agents (e.g., peptides, antibodies, peptidomimetics, small molecules (organic or inorganic) or other drugs) which reduce DUX4 expression, for example, by activating the Wnt/β-catenin pathway.

The term “candidate agent” is used herein to mean any agent that is being examined for ability to modulate the activity or expression of one or more members of the Wnt/β-catenin pathway in skeletal muscle cells, and particularly in skeletal muscle cells expressing an FSHD phenotype in culture. Although the method generally is used as a screening assay to identify previously unknown molecules that can act as a therapeutic agent, the screening described herein can also be used to confirm that an agent known to have such activity, in fact has the activity, for example, in standardizing the activity of the therapeutic agent. A candidate agent can be any type of molecule, including, for example, a peptide, a peptidomimetic, a polynucleotide, or a small organic molecule, that one wishes to examine for the ability to modulate a desired activity, such as, for example, decreasing DUX4 expression and/or activity. It will be recognized that the methods described herein are readily adaptable to a high throughput format and, therefore, the methods are convenient for screening a plurality of test agents either serially or in parallel. The plurality of test agents can be, for example, a library of test agents produced by a combinatorial method library of test agents. Methods for preparing a combinatorial library of molecules that can be tested for therapeutic activity are well known in the art and include, for example, methods of making a phage display library of peptides, which can be constrained peptides (see, for example, U.S. Pat. Nos. 5,622,699; 5,206,347; Scott and Smith, Science 249:386-390, 1992; Markland et al., Gene 109:1319, 1991; each of which is incorporated herein by reference in their entireties); a peptide library (U.S. Pat. No. 5,264,563, which is incorporated herein by reference); a peptidomimetic library (Blondelle et al., Trends Anal. Chem. 14:8392, 1995; a nucleic acid library (O'Connell et al., supra, 1996; Tuerk and Gold, supra, 1990; Gold et al., supra, 1995; each of which is incorporated herein by reference in their entireties); an oligosaccharide library (York et al., Carb. Res., 285:99128, 1996; Liang et al., Science, 274:1520-1522, 1996; Ding et al., Adv. Expt. Med. Biol., 376:261-269, 1995; each of which is incorporated herein by reference in their entireties); a lipoprotein library (de Kruif et al., FEBS Lett., 399:232-236, 1996, which is incorporated herein by reference in their entireties); a glycoprotein or glycolipid library (Karaoglu et al., J. Cell Biol., 130:567-577, 1995, which is incorporated herein by reference); or a chemical library containing, for example, drugs or other pharmaceutical agents (Gordon et al., J. Med. Chem., 37:1385-1401, 1994; Ecker and Crooke, Bio/Technology, 13:351-360, 1995; each of which is incorporated herein by reference in their entireties).

Accordingly, the term “agent” as used herein in the context of screening means any compound or substance such as, but not limited to, a small molecule, nucleic acid, polypeptide, peptide, drug, ion, etc. An “agent” can be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In some embodiments, the nucleic acid is DNA or RNA, and nucleic acid analogues, for example can be PNA, pcPNA and LNA. A nucleic acid may be single or double stranded, and can be selected from a group comprising; nucleic acid encoding a protein of interest, oligonucleotides, PNA, etc. Such nucleic acid sequences include, for example, but are not limited to, nucleic acid sequence encoding proteins that act as transcriptional repressors, antisense molecules, ribozymes, small inhibitory nucleic acid sequences, for example but not limited to RNAi, shRNAi, siRNA, micro RNAi (mRNAi), antisense oligonucleotides etc. A protein and/or peptide agent or fragment thereof, can be any protein of interest that activates the Wnt/β-catenin signaling pathway, for example, but not limited to; mutated proteins; therapeutic proteins; truncated proteins, wherein the protein is normally absent or expressed at lower levels in the cell. Proteins of interest can be selected from a group comprising; mutated proteins, genetically engineered proteins, peptides, synthetic peptides, recombinant proteins, chimeric proteins, antibodies, humanized proteins, humanized antibodies, chimeric antibodies, modified proteins and fragments thereof.

In certain embodiments, the candidate agent is a small molecule having a chemical moiety. Such chemical moieties can include, for example, unsubstituted or substituted alkyl, aromatic, or heterocyclyl moieties and typically include at least an amine, carbonyl, hydroxyl or carboxyl group, frequently at least two of the functional chemical groups, including macrolides, leptomycins and related natural products or analogues thereof. Candidate agents can be known to have a desired activity and/or property, or can be selected from a library of diverse compounds. Also included as candidate agents are pharmacologically active drugs, genetically active molecules, etc. Such candidate agents of interest include, for example, chemotherapeutic agents, hormones or hormone antagonists, growth factors or recombinant growth factors and fragments and variants thereof. Exemplary of pharmaceutical agents suitable for use with the screening methods described herein are those described in, “The Pharmacological Basis of Therapeutics,” Goodman and Gilman, McGraw-Hill, New York, N.Y., (1996), Ninth edition, under the sections: Water, Salts and Ions; Drugs Affecting Renal Function and Electrolyte Metabolism; Drugs Affecting Gastrointestinal Function; Chemotherapy of Microbial Diseases; Chemotherapy of Neoplastic Diseases; Drugs Acting on Blood-Forming organs; Hormones and Hormone Antagonists; Vitamins, Dermatology; and Toxicology, all of which are incorporated herein by reference in their entireties. Also included are toxins, and biological and chemical warfare agents, for example see Somani, S. M. (Ed.), “Chemical Warfare Agents,” Academic Press, New York, 1992), the contents of which is herein incorporated in its entirety by reference. Candidate agents, such as chemical compounds, can be obtained from a wide variety of sources including libraries of synthetic or natural compounds, such as small molecule compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and can be used to produce combinatorial libraries. Known pharmacological agents can be subjected to directed or random chemical modifications, such as acylation, alkylation, esterification, amidification, etc. to produce structural analogs. Synthetic chemistry transformations and protecting group methodologies (protection and deprotection) useful in synthesizing the candidate compounds for use in the screening methods described herein are known in the art and include, for example, those such as described in R. Larock (1989) Comprehensive Organic Transformations, VCH Publishers; T. W. Greene and P. G. M. Wuts, Protective Groups in Organic Synthesis, 2nd ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof, the contents of each of which are herein incorporated in their entireties by reference. Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al (1994) Angew. Chem. Int. Ed. Engl. 33:2061; and Gallop et al. (1994) J. Med. Chem. 37:1233, the contents of each of which are herein incorporated in their entireties by reference. Libraries of candidate agents can also, in some embodiments, be presented in solution (e.g., Houghten (1992), Biotechniques 13:412-421), or on beads (Lam (1991), Nature 354:82-84), chips (Fodor (1993) Nature 364:555-556), bacteria (Ladner, U.S. Pat. No. 5,223,409), spores (Ladner U.S. Pat. No. 5,223,409), plasmids (Cull et al. (1992) Proc Natl Acad Sci USA 89:1865-1869) or on phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.), the contents of each of which are herein incorporated in their entireties by reference. The test compounds or candidate agents can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145). Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233. Libraries of compounds can be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.). The methods described herein further pertain to novel agents identified by the above-described screening assays. With regard to intervention, any treatments which modulate DUX4 expression and/or activity (e.g., via the Wnt/β-catenin pathway) should be considered as candidates for human therapeutic intervention.

In one embodiment, a screening assay is a cell-based assay comprising contacting a cell (e.g., a myotube) in culture with a candidate agent and determining the ability of the candidate agent to modulate (e.g., induce or inhibit) DUX4 activity and/or expression. The screening assays described herein can be used alone, or in combination with at least one other screening assay as described herein.

It is understood that the foregoing description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

### EXAMPLES

### Example 1

### Myotubes Generated from FSHD-Muscle Biopsies Undergo Sporadic Apoptosis and Enable the Identification of Chemicals and Pathways that Reduce DUX4 Expression

Using a novel method of myoblast differentiation, the inventors demonstrate that primary FSHD-myotubes undergo apoptosis initiated by myotube-specific activation of DUX4. siRNAs targeting DUX4 and chemical inhibitors of apoptosis prevent myotube toxicity Inhibitors of Rho associated kinase, or activation of Wnt/β-catenin signaling in the differentiated myotubes prevent DUX4 expression and its toxic effects. Finally, we show that the FSHD2-causing gene SMCHD1, and the maintenance methyltransferase DNMT1 contribute to DUX4 repression in FSHD1-myotubes demonstrating that these genes potentially contribute to disease severity and penetrance. Together these findings inform pathogenic mechanisms and reveal druggable targets for therapy development.

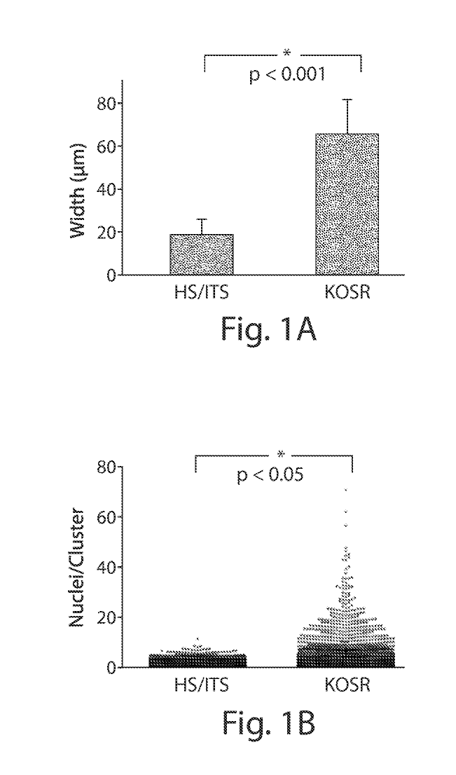

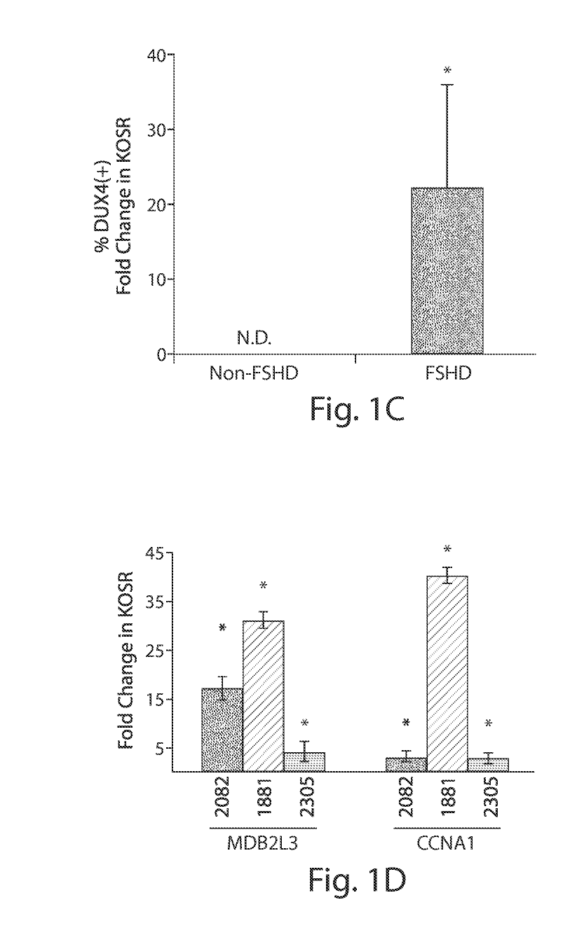

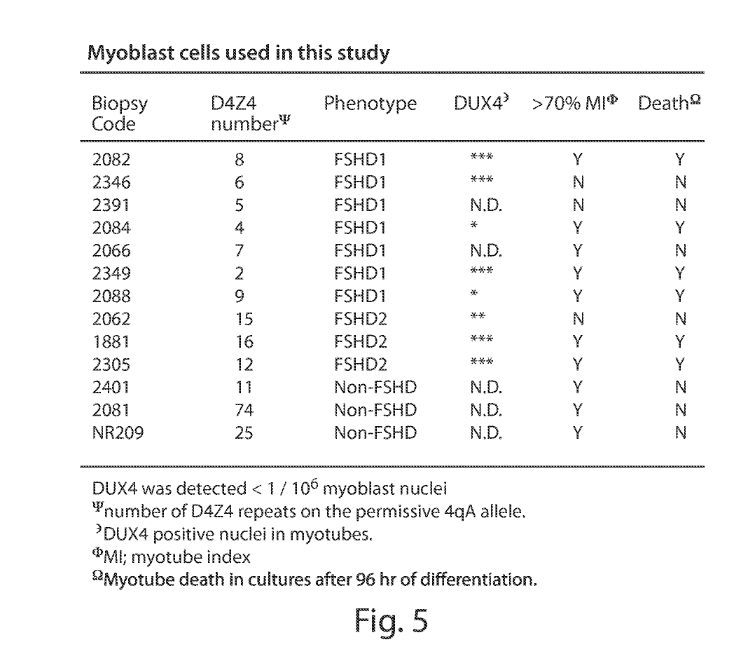

Differentiation of cultured human myoblasts is initiated by mitogen depletion that is generally accomplished by reducing serum concentrations in the medium and supplementing with insulin, transferrin and selenium¹⁷. The inventors found that growth factor reduction without serum starvation by using 20% serum replacement preparation (e.g., knock out serum replacer (KOSR)), improved myoblast differentiation and generated a hypertrophic myotube phenotype¹⁸ (data not shown) characterized by larger tubes containing more nuclei (FIGS. 1A-B). Applying this differentiation protocol to FSHD-muscle derived myoblasts resulted in an increase in the percentage of myotube nuclei expressing DUX4 when compared to the same cells differentiated using conventional conditions (HS/ITS) (data not shown). That is, FSHD-derived myoblasts expressed an FSHD phenotype that was not promoted when such cells were cultured in a standard differentiation-promoting medium. Regardless of the differentiation protocol, DUX4 was exclusively expressed in MHC(+) myotubes. To account for variable myotube formation between different cell cultures, the percentage of DUX4(+) nuclei is expressed as a fraction of nuclei incorporated into MHC(+) myotubes (FIG. 1C). Using this metric DUX4 protein was detected in 0.5%-2.0% of myotubes when myoblasts were differentiated using conventional HS/ITS conditions and 3.0%-16.5% when differentiated using KOSR (FIG. 1C and data not shown). Expression from two DUX4-activated genes, MDB2L3, and CCNA1¹⁹, was also increased in all FSHD cell lines tested (FIG. 1D). Since the percentage of DUX4(+) nuclei varied between different cell lines, the inventors investigated DUX4 immunoreactivity in myotubes generated from a number of FSHD-affected muscle biopsies (FIG. 5). The inventors chose myoblast cell lines that formed myotubes with the highest percentage of DUX4(+) nuclei for subsequent studies. These included FSHD1(2349), which contained two D4Z4 units, FSHD2(2305) with 12 units, and FSHD2(1881) with 16 units.

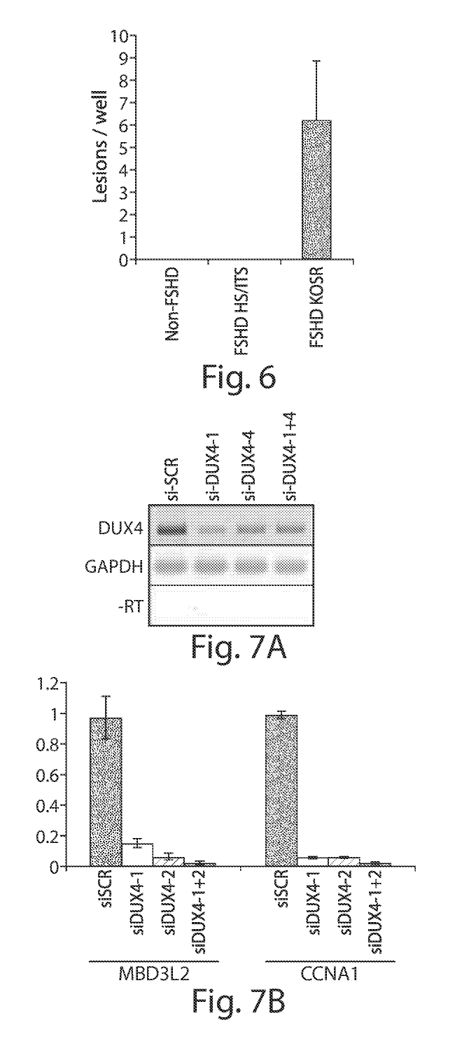

In addition to increased DUX4 expression, FSHD myoblasts induced to differentiate in KOSR-containing medium developed cytopathic lesions that appeared as retracted myotubes surrounding a lawn of myoblasts (data not shown) and the lesions progressed to complete loss of myotubes in the cultures after 72-96 hours of differentiation. The myotubes adjacent to lesions incorporated DAPI indicating a loss in cell membrane integrity and were only present in FSHD-myoblast cultures differentiated in KOSR (FIG. 6). DUX4 protein was consistently detected within 500 μm of retracted myotubes and infrequently detected outside of the lesions suggesting the cytopathology was directly related to DUX4 expression (data not shown). Cytopathic lesions were present in differentiated cultures of myoblasts from most of our FSHD-derived samples, including cells from people with either FSHD1 or FSHD2 (FIG. 5).

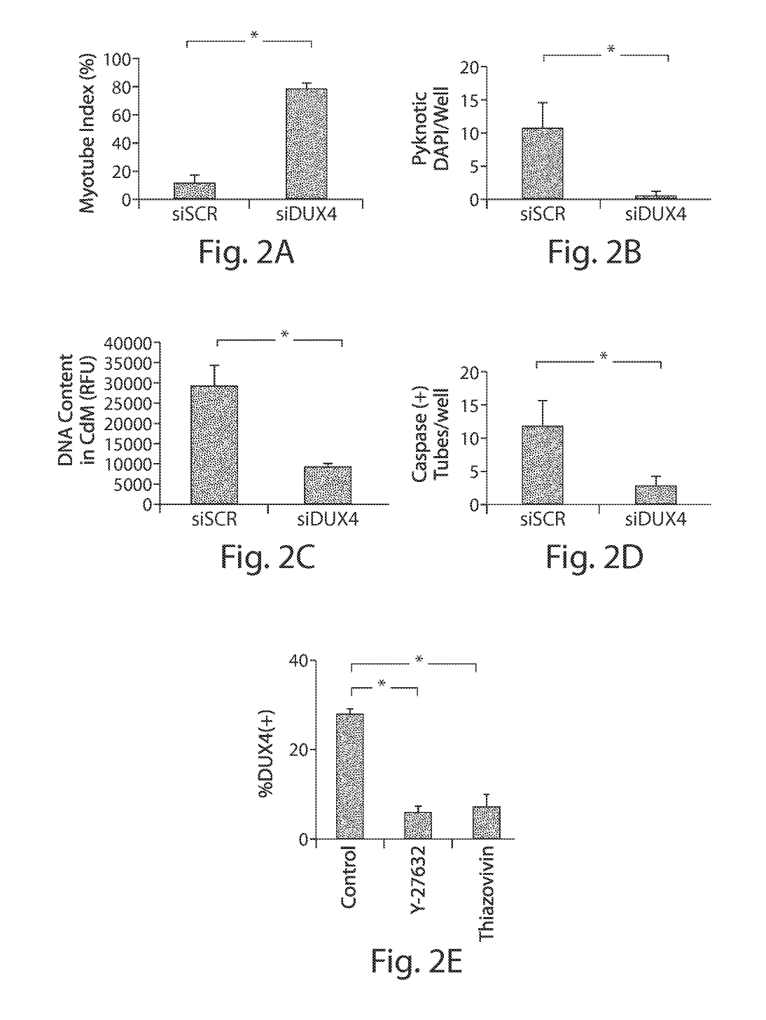

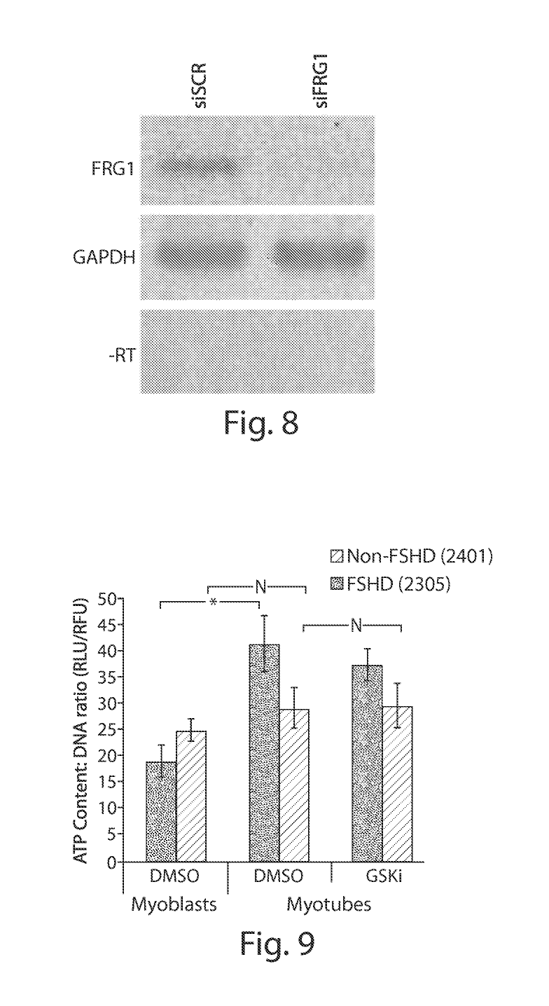

DUX4(+) nuclei showed chromatin condensation and contracted myotubes contained clusters of TUNEL(+) pyknotic nuclei that were immunoreactive for activated caspase-3 suggesting an apoptotic mechanism (data not shown). After 96 hrs of differentiation, FSHD-cultures had widespread loss of myotubes, that was reversed by transfection with a siRNA targeting DUX4¹⁹ (FIGS. 7A-7B and data not shown). Because toxicity was myotube specific, myoblasts transfected with DUX4 siRNA produced myotubes with a higher myotube index (FIG. 2A), lower numbers of pyknotic nuclei (FIG. 2B), lower DNA content in the medium (FIG. 2C) and fewer myotubes with activated caspase-3 immunoreactivity (FIG. 2D). Similar results were achieved by transfection of cells with a siRNA targeting p53 transcripts supporting previous studies performed in mouse cells and tissues (FIG. 2E)²⁰. FSHD related gene 1 (FRG1) has also been implicated in FSHD pathology due to its location adjacent to the D4Z4 array on chromosome 4²¹, and the existence of transgenic mice that exhibit myopathology when the gene is overexpressed in muscle tissue²². However, reduction of FRG1 transcripts did not ameliorate myotube toxicity (data not shown and FIG. 8).

Using this cell culture model of FSHD-specific pathology the inventors tested several chemicals that were hypothesized to abrogate DUX4 toxicity and thus be candidates for FSHD therapies. Oxidative stress has been a frequently cited feature of FSHD pathology because of a screen that showed increased expression of genes in oxidative response pathways²³, and because dysregulation of oxidative response pathways was also identified in mouse myoblasts overexpressing DUX4²⁴. The inventors treated FSHD myotubes with the antioxidants β-mercapto-ethanol and ascorbic acid but observed DUX4-dependent myotube apoptosis despite treatments at concentrations that rescued mouse myoblasts under conditions of DUX4 overexpression²⁴ (data not shown). Furthermore, several lines of evidence suggest that p53 is a component of the final common pathway of DUX4-mediated apoptosis^{20,25} (FIGS. 2C-E and data not shown) so the inventors tested the effects of a small library of anti-apoptotic reagents for their ability to prevent DUX4-mediated myotube death. Treatment of FSHD-myotubes with the p53 inhibitor pifithrin-α²⁶ or Arsenic trioxide, a chemotherapeutic agent that prevents p53 activation by disrupting PML nuclear bodies, efficiently prevented DUX4-mediated myotube death (data not shown). Rho kinase inhibitors Thiazovivin and Y-27632²⁷ also prevented DUX4-mediated myotube death but unlike chemicals that inhibit p53 activity, the reduction in myotube apoptosis could be attributed to a 10-fold reduction in the number of nuclei expressing DUX4 (FIGS. 2E, 2F).

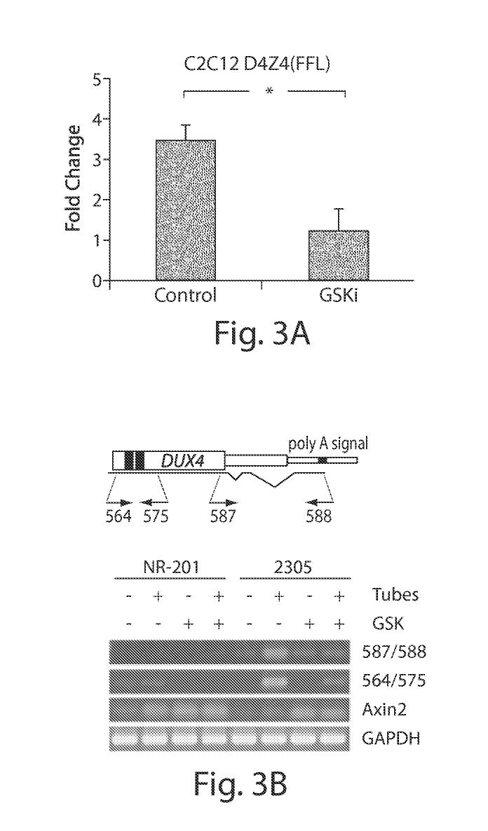

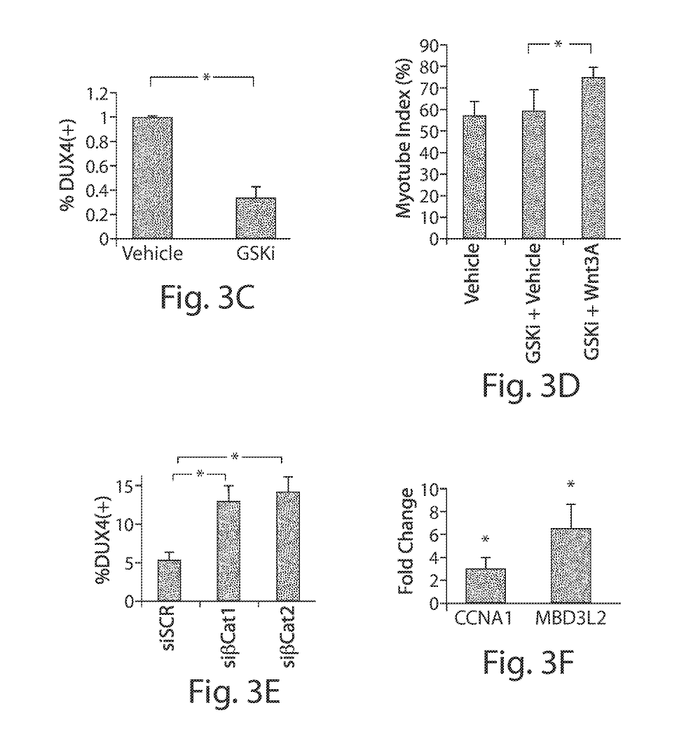

The Wnt/β-catenin signaling pathway has been implicated in FSHD pathology because of its role in muscle development and facial muscle organization²⁸, and because people with mutations in the Wnt receptors frizzled-4 and LRP5, have a specific retinal pathology indistinguishable from that present in some patients with FSHD^{29,30}. The inventors used a previously characterized clone of C2C12 cells containing a DUX4 reporter to measure the effect of Wnt/β-catenin pathway activation on DUX4 transcription. Treatment of these cells or primary human cells (normal(NR201) and FSHD2(2305)) with an inhibitor of glycogen synthase kinase 3β (GSKi) to activate Wnt signaling, reduced the characteristic upregulation of DUX4 expression during myotube formation³¹ (FIGS. 3A, 3B). FSHD myoblasts differentiated in the presence of GSKi showed a greater than 50% reduction in the number of DUX4(+) nuclei in myotubes (FIG. 3C); however, GSKi treatment alone did not prevent myotube apoptosis (FIG. 9). Further stimulation of the Wnt/β-catenin signaling by adding a combination of recombinant Wnt3a and GSKi prevented DUX4-mediated apoptosis in FSHD-myotubes and reduced the percentage of DUX4 expressing nuclei from 10% to 2% (FIG. 3D). Conversely, reducing Wnt signaling activity by reduction of the downstream target of GSK3-β, β-catenin with siRNAs resulted in a 12 fold increase in the number of DUX4(+) nuclei (FIG. 3E) with concomitant activation of DUX4 target genes CCNA1 and MBD3L2 (FIG. 3F). These data demonstrate that Wnt/β-catenin signaling negatively regulates DUX4 expression in FSHD-myotubes.

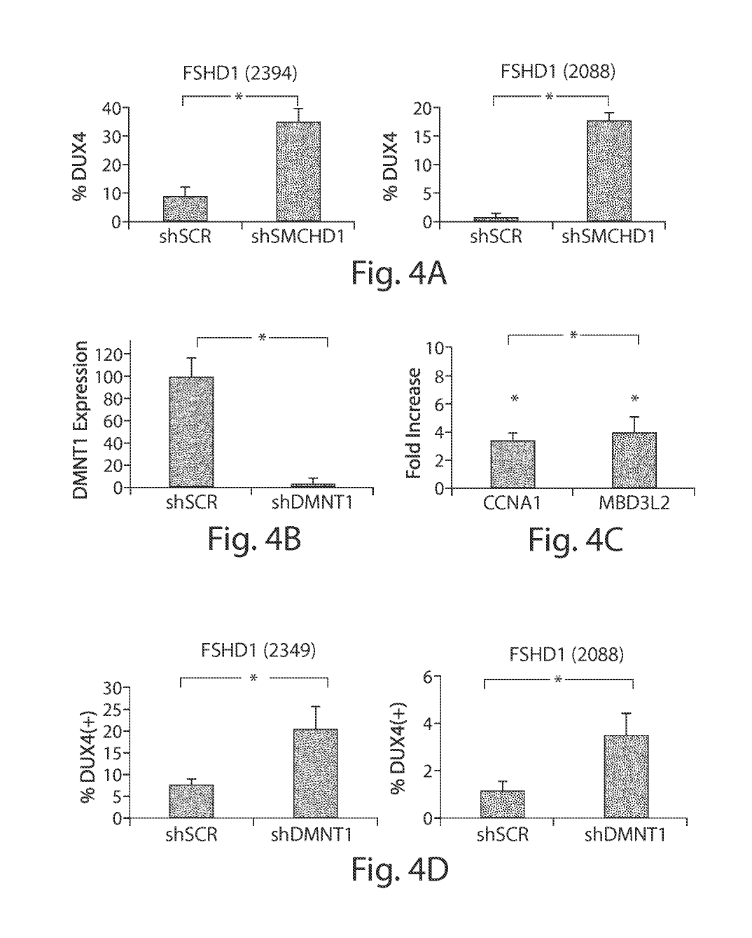

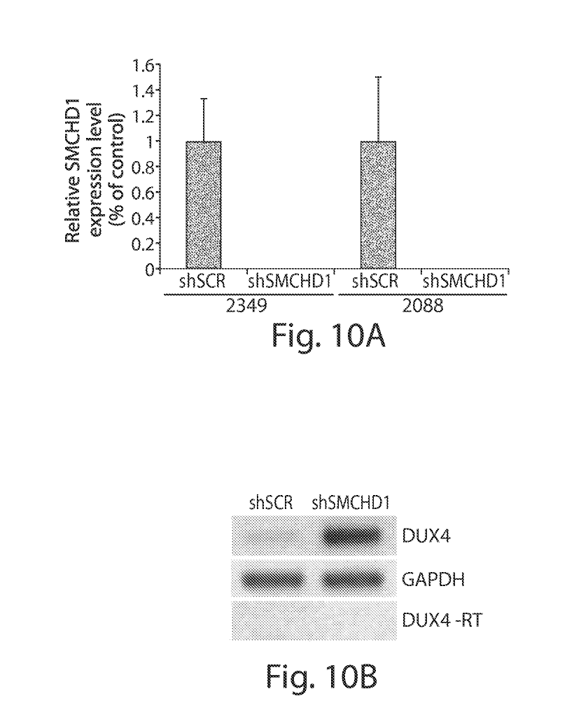

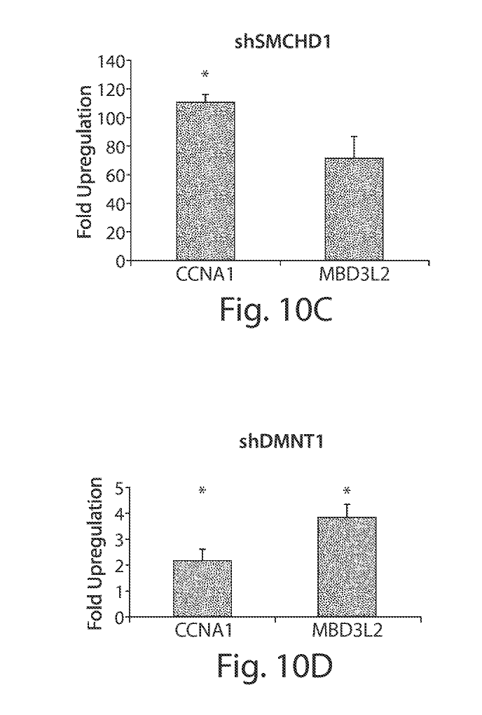

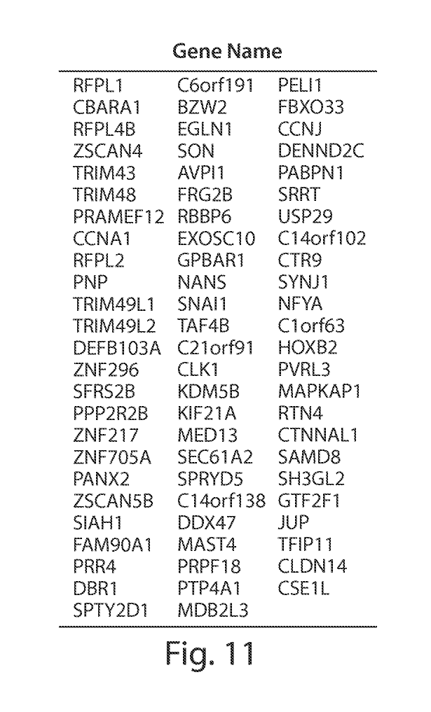

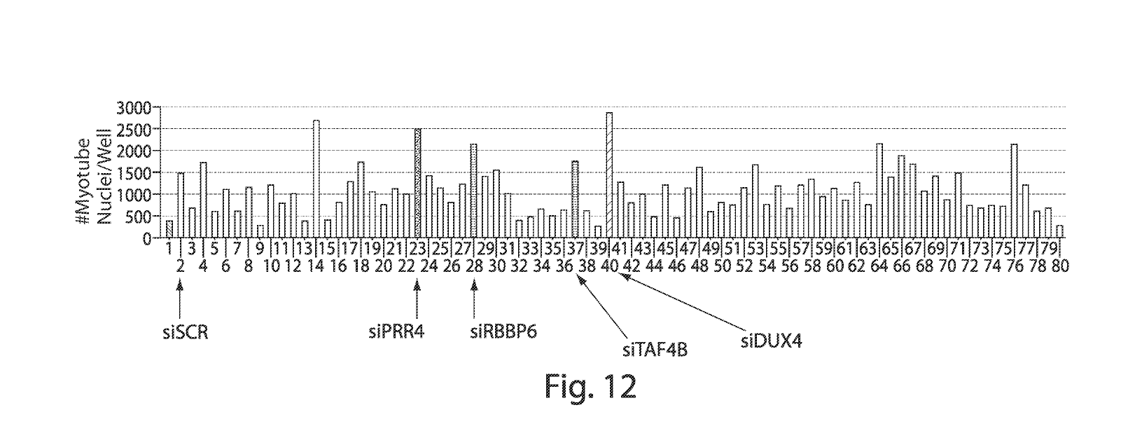

Mutations in structural maintenance of chromatin hinge domain 1 (SMCHD1) have recently been shown to cause FSHD2 through epigenetic changes that relax D4Z4 chromatin and result in DUX4 expression¹¹. SMCHD1 is a chromatin modifier involved in X chromosome inactivation³² and was previously identified in a mouse mutagenesis screen because mice carrying Smchd1 or Dnmt1 mutations had higher percentages of erythrocytes that express GFP from a partially silenced (D4Z4-like) array of GFP reporters³³. The inventors hypothesized that SMCHD1 limits DUX4 expression from short arrays found in FSHD1 patients through an epigenetic mechanism. SMCHD1 levels were reduced in two FSHD1 cell lines containing a single FSHD-permissive D4Z4 array using a shRNA expressed from a lentiviral vector. Myotubes derived from these cells showed increased transcript levels of DUX4, and DUX4 activated genes (FIGS. 10A-10D). Reduction of SMCHD1 transcripts resulted in an increased percentage of DUX4(+) nuclei (FIG. 4A and data not shown) which induced DUX4-dependent myotube death more rapidly than controls containing a scrambled shRNA (shSCR) (data not shown).

DNMT1 mutations have not been described in FSHD2 patients, however reduction of DNMT1 transcripts in normal human myoblasts resulted in variegated DUX4 expression (data not shown) and DNMT1 transcript reduction in FSHD1-derived myotubes resulted in a higher frequency of DUX4 expression (FIGS. 4B, 4D) and expression of DUX4 target genes (FIG. 4C). These results indicate that both DNMT1 and SMCHD1 activities suppress expression from relaxed or partially relaxed D4Z4 arrays and demonstrate the potential of expression levels from these genes to alter disease severity and penetrance.

Here the inventors validate a human cell culture model that features intrinsic DUX4-dependent myotube toxicity in myotubes generated from FSHD-muscle biopsies. Using this model the inventors demonstrate that inhibitors of apoptosis can prevent DUX4-mediated myotube toxicity whereas inhibition of rho-associated kinase or stimulation of the Wnt/β-catenin pathway can reverse the FSHD phenotype in culture. Furthermore, the inventors demonstrate that genes involved in epigenetic silencing of macrosatellite arrays can influence the level of DUX4 expression even when the array length is short and the chromatin conformation appears to be open. Thus, the inventors have identified a new set of potentially therapeutic targets for FSHD therapies. Finally the inventors provide a proof of principle for refining disease mechanisms and testing therapeutic candidates highlighting the potential for the use of this platform for drug discovery.

### Methods

Cell Lines and Cell Culture Reagents.

Primary human myoblasts were obtained by written informed consent through the Fields Center at the University of Rochester cell bank. Myoblasts were grown in F10 medium (INVITROGEN, Carlsbad, Calif.) supplemented with 20% fetal bovine serum (FBS; THERMO SCIENTIFIC (HYCLONE), and 50 U/50 μg penicillin/streptomycin (Pen-Strep; INVITROGEN), 10 ng bFGF (INVITROGEN), and 1 μM dexamethasone (SIGMA ALDRICH; St. Louis, Mich.)⁹. Differentiation was induced using F10 medium supplemented with 1% equine serum and ITS supplement (insulin 0.1%, 0.000067% sodium selenite, 0.055% transferrin; INVITROGEN), or in DMEM:F12 (1:1) with 3.151 g/L glucose, 10 mM non-essential amino acids (INVITROGEN), 100 mM sodium pyruvate, 20% knockout serum replacer (INVITROGEN, #10828010). Pharmacological inhibitor of glycogen synthase kinase-3-β (GSKi) was used at a final concentration of 3 μM (CHIR99021, STEMGENT; Cambridge. Mass.). Recombinant human Wnt3A was used at a concentration of 250 ng/mL, and confirmed to be bioactive prior to use in the assays described herein.

Immunofluorescent Microscopy.

Immunofluorescent staining for DUX4 was performed using the E5-5 rabbit monoclonal antibody to the C-terminus of DUX4 ³⁴. Staining was performed by fixing the cells in 4% paraformaldehyde for 10 mins at room temperature, washing with PBS, permeablizing with 0.5% TritonX-100 in PBS for 10 mins at room temperature. The cells were then washed again in PBS and incubated overnight in a 1:10 dilution of the E5-5 antibody. The following day, the cells were washed 3×5 min in PBS and stained using a fluorescent conjugated secondary goat-anti rabbit secondary antibody for 1-2 hr at room temperature. Following a final wash, the cells were counterstained with 4′,6-Diamidino-2-phenylindole dihydrochloride (DAPI, SIGMA ALDRICH), and visualized by microscopy using UV illumination. Where applicable, the cultures were co-stained with other antibodies including the myosin heavy chain (1:1000 dilution MF20 clone; R&D systems), anti-caspase-3 (CELL SIGNALING TECHNOLOGY, Danvers, Mass.).

Image Quantification.

To quantify DUX4 expression, the Nikon Ti Eclipse microscope was programmed using the Nikon Elements software to take 36 pictures at random on a 35 mm culture dish, or to scan an entire well of a 384 well dish. These images were then manually inspected and DUX4 (+) nuclei counted. To determine the myotube index, the Nikon Elements software was used to create binary masks of the DAPI and MHC channels for each image. This technique created a border around each nucleus or cluster of nuclei which could then be used to measure the sum pixel intensity from the DAPI channel. The average sum pixel intensity for single nuclei from each image was determined, and used to calculate the number of nuclei within clusters. By intersecting the binary images from the DAPI and MHC channels the inventors could exclude the nuclei that were outside of MHC(+) myotubes, and create a myotube fusion index. The myotube fusion index was compared to manual counts to confirm accuracy of the calculations.

DNA Cloning.

The D4Z4 regulatory region controlling the expression of DUX4³¹ was subcloned upstream of the open reading from of the pGL3-basic vector (pGL3) to make D4Z4(FFL). The D4Z4(FFL) was excised using XbaI/SpeI restriction enzymes and ligated into the SpeI site of a 3^{rd} generation lentiviral vector multiple cloning site (pRRL-sinc-PPT-MCS; Gift from Dr. Grant Trobridge).

Luciferase Assays.

Cells transduced with D4Z4(FFL) lentivirus vector were seeded in a 96 well dish. Thirty minutes prior to the assay, Hoescht Dye was added to the cell culture medium to a final concentration of 5 μm, and 30 min later the plate was analyzed for DNA content by measuring fluorescence at 355 nm using a multiwell plate reader (ENVISION; PERKIN-ELMER). An equal volume of STEADY-GLO (PROMEGA) was added to each well, and transferred to an opaque dish. Luminescence was read using the plate reader, and normalized to DNA content for each well.

Cell Viability Assays.

Hoescht Dye was added to cells in culture for 30 minutes at a final concentration of 5 μM. Following detachment with trypsin a 50 μL aliquot of the cells was assayed for fluorescence at 355 nm to give an absorbance value for DNA content. An equal volume of CELL TITRE GLO (PROMEGA) was then added to the aliquot, and luminescence was measured. The luminescent value was normalized to the absorbance value for DNA content.

Semi-Quantitative PCR.

RNA was isolated by TRIZOL extraction according to the manufacturers protocol (INVITROGEN), except that the final ethanol precipitate was resuspended 1×DNase I buffer and 5 U DNase I for 15 mins and the RNA was extracted with phenol/chloroform and precipitated with ethanol using RNase-free glycogen (NEW ENGLAND BIOLABS) as a carrier. One μg of RNA was converted to DNA by reverse transcription using Oligo dT primers and the SUPERSCRIPT III 1st strand cDNA synthesis kit. The reverse transcription reaction was incubated first at 65° for 5 min, followed by 45° for 45 min, 50° for 15 min, and 55° for 15 min, and 75° for 15 min in a total of 50 pls. PCR for DUX4 was performed using the PCRX enhancer system for GC rich sequences (INVITROGEN) and Taq polymerase (NEW ENGLAND BIOLABS). Cycling conditions: 35 cycles of 94° C.×30 s, 55° C.×30 s, 68° C.×120 s. To control for genomic DNA contamination, a no-RT control was always included for each PCR sample. Primer Sequences:

587:
GCTGGAAGCACCCCTCAGCGAGGAA;
588:
GAATTCCATGGCGTCGTTTACTTTGACCAACAAGAA;
564:
CGATGCCCGGGTACGGGTTCCGCTCAAAGC;
575:
GCAGTGCACAGTCCGGCTGAGGTGCACGG;
GAPDHF:
ATCTTCCAGGAGCGAGATCC;
GAPDHR:
ACCACTGACACGTTGGCAGT

siRNA Transfections.

siRNA for DUX4 was custom synthesized using previously published sequences³⁴ (THERMO SCIENTIFIC). 5 μL of 20 μM stock was resuspended in 250 μL OPTIMEM (INVITROGEN) and incubated for 15 mins with 6 μL LIPOFECTAMINE RNAiMaX (INVITROGEN). The mixture was added to myoblasts that had been seeded 24 hrs prior at 50,000 cells/cm². Twenty-four hrs later, the medium was changed to KOSR differentiation medium and RNA from the cells was assayed by PCR and cells fixed in formaldehyde were assessed by immunofluorescent microscopy for DUX4 reduction. Conditions were scaled down to be performed in 384 well dishes. In these experiments, 1.8 μL of LIPOFECTAMINE RNAiMax was added to 100 μL OPTIMEM. 1 μL siRNA was added to 19 μL of liposome-containing OPTIMEM. Five μL of the siRNA mixture was used per well of a 384 well dish. siRNA sequences: siDUX4-1:5′-r(GAUGAUUAGUUCAGAGAUA)d(TT)-3′. siDUX4-2:5′-r(GCGCAACCUCUCCUAGAAA)d(TT)-3′). For a non-targeting control the inventors used the Non-targeting control siRNA #1 (THERMO SCIENTIFIC). SILENCER SELECT siRNAs to β-catenin, p53, FRG1 and a corresponding scrambled control were purchased from INVITROGEN (#s437, #s606, #s5367 SILENCER SELECT Negative Control 1 respectively).

### Reduction of SMCHD1 and DNMT1 Transcript Levels in Myoblasts.

SMCHD1 and DNMT1 transcripts were targeted for degradation using lentiviral vectors expressing short hairpin RNAs from a CMV promoter and linked to a puromycin selection cassette by an internal ribosome entry site (IRES). Five different pGIPZ (OPEN BIOSYSTEMS, Huntsville, Ala.) vectors were purchased and each was tested in normal human myoblasts for the effect on SMCHD1 and DMNT1 transcripts by quantitative PCR.

Statistics.

For experiments in which two means were being compared, statistical significance was determined using a two-tailed Student's t-test. When more than two means were being compared, a one-way analysis of variance was performed. When counts were being compared, for example when counting DUX4(+) nuclei, a Fisher exact test was used. When comparing the distribution of nuclei in clusters (FIG. 1B), statistical significance was determined using the Mann-Whitney test. Statistical data was processed using GRAPHPAD software (PRISM).

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

All of the references cited herein are incorporated by reference. Aspects of the disclosure can be modified, if necessary, to employ the systems, functions, and concepts of the above references and application to provide yet further embodiments of the disclosure. These and other changes can be made to the disclosure in light of the detailed description.

Specific elements of any foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

### REFERENCES

1. Tawil, R. Facioscapulohumeral muscular dystrophy. Neurotherapeutics 5, 601-6 (2008).
2. Wijmenga, C. et al. Location of facioscapulohumeral muscular dystrophy gene on chromosome 4. Lancet 336, 651-3 (1990).
3. van Deutekom, J. C. et al. FSHD associated DNA rearrangements are due to deletions of integral copies of a 3.2 kb tandemly repeated unit. Hum Mol Genet 2, 2037-42 (1993).
4. Wijmenga, C. et al. Chromosome 4q DNA rearrangements associated with facioscapulohumeral muscular dystrophy. Nat Genet 2, 26-30 (1992).
5. van Overveld, P. G. et al. Hypomethylation of D4Z4 in 4q-linked and non-4q-linked facioscapulohumeral muscular dystrophy. Nat Genet 35, 315-7 (2003).
6. Dixit, M. et al. DUX4, a candidate gene of facioscapulohumeral muscular dystrophy, encodes a transcriptional activator of PITX1. Proc Natl Acad Sci USA 104, 18157-62 (2007).
7. Gabriels, J. et al. Nucleotide sequence of the partially deleted D4Z4 locus in a patient with FSHD identifies a putative gene within each 3.3 kb element. Gene 236, 25-32 (1999).
8. Lemmers, R. J. et al. A unifying genetic model for facioscapulohumeral muscular dystrophy. Science 329, 1650-3 (2010).
9. Snider, L. et al. RNA transcripts, miRNA-sized fragments and proteins produced from D4Z4 units: new candidates for the pathophysiology of facioscapulohumeral dystrophy. Hum Mol Genet 18, 2414-30 (2009).
10. de Greef, J. C. et al. Common epigenetic changes of D4Z4 in contraction-dependent and contraction-independent FSHD. Hum Mutat 30, 1449-59 (2009).
11. Lemmers, R. J. L. F. et al. Digenic inheritance of an SMCHD1 mutation and an FSHD-permissive D4Z4 allele causes facioscapulohumeral muscular dystrophy type 2 Nat Genet in press (2012).
12. Snider, L. et al. Facioscapulohumeral dystrophy: incomplete suppression of a retrotransposed gene. PLoS Genet 6, e1001181 (2010).
13. Barro, M. et al. Myoblasts from affected and non-affected FSHD muscles exhibit morphological differentiation defects. J Cell Mol Med 14, 275-89 (2010).
14. Homma, S. et al. A unique library of myogenic cells from facioscapulohumeral muscular dystrophy subjects and unaffected relatives: family, disease and cell function. Eur J Hum Genet 20, 404-10 (2012).
15. Tsumagari, K. et al. Gene expression during normal and FSHD myogenesis. BMC Med Genomics 4, 67 (2011).
16. Ehrlich, M. & Lacey, M. Deciphering transcription dysregulation in FSH muscular dystrophy. J Hum Genet 57, 477-84 (2012).
17. Blau, H. M. & Webster, C. Isolation and characterization of human muscle cells. Proc Natl Acad Sci USA 78, 5623-7 (1981).
18. Semsarian, C., Sutrave, P., Richmond, D. R. & Graham, R. M. Insulin-like growth factor (IGF-I) induces myotube hypertrophy associated with an increase in anaerobic glycolysis in a clonal skeletal-muscle cell model. Biochem J 339 (Pt 2), 443-51 (1999).
19. Geng, L. N. et al. DUX4 Activates Germline Genes, Retroelements, and Immune Mediators: Implications for Facioscapulohumeral Dystrophy. Dev Cell 22, 38-51 (2012).
20. Wallace, L. M. et al. DUX4, a candidate gene for facioscapulohumeral muscular dystrophy, causes p53-dependent myopathy in vivo. Ann Neurol 69, 540-52 (2011).
21. van Deutekom, J. C. et al. Identification of the first gene (FRG1) from the FSHD region on human chromosome 4q35. Hum Mol Genet 5, 581-90 (1996).
22. Gabellini, D. et al. Facioscapulohumeral muscular dystrophy in mice overexpressing FRG1. Nature 439, 973-7 (2006).
23. Winokur, S. T. et al. Facioscapulohumeral muscular dystrophy (FSHD) myoblasts demonstrate increased susceptibility to oxidative stress. Neuromuscul Disord 13, 322-33 (2003).
24. Bosnakovski, D. et al. An isogenetic myoblast expression screen identifies DUX4-mediated FSHD-associated molecular pathologies. Embo J 27, 2766-79 (2008).
25. Kowaljow, V. et al. The DUX4 gene at the FSHD1A locus encodes a pro-apoptotic protein. Neuromuscul Disord 17, 611-23 (2007).
26. Komarov, P. G. et al. A chemical inhibitor of p53 that protects mice from the side effects of cancer therapy. Science 285, 1733-7 (1999).
27. Uehata, M. et al. Calcium sensitization of smooth muscle mediated by a Rho-associated protein kinase in hypertension. Nature 389, 990-4 (1997).
28. Tajbakhsh, S. et al. Differential activation of Myf5 and MyoD by different Wnts in explants of mouse paraxial mesoderm and the later activation of myogenesis in the absence of Myf5. Development 125, 4155-62 (1998).
29. Robitaille, J. et al. Mutant frizzled-4 disrupts retinal angiogenesis in familial exudative vitreoretinopathy. Nat Genet 32, 326-30 (2002).
30. Fitzsimons, R. B. Retinal vascular disease and the pathogenesis of facioscapulohumeral muscular dystrophy. A signalling message from Wnt? Neuromuscul Disord 21, 263-71 (2011).
31. Block, G. J. et al. Asymmetric bidirectional transcription from the FSHD-causing D4Z4 array modulates DUX4 production. PLoS ONE 7, e35532 (2012).
32. Blewitt, M. E. et al. SmcHD1, containing a structural-maintenance-of-chromosomes hinge domain, has a critical role in X inactivation. Nat Genet 40, 663-9 (2008).
33. Blewitt, M. E. et al. An N-ethyl-N-nitrosourea screen for genes involved in variegation in the mouse. Proc Natl Acad Sci USA 102, 7629-34 (2005).
34. Geng, L. N., Tyler, A. E. & Tapscott, S. J. Immunodetection of human double homeobox 4. Hybridoma (Larchmt) 30, 125-30 (2011).

1. A method for inducing skeletal muscle cells from an individual with Facioscapulohumeral Muscular Dystrophy (FSHD) to express an FSHD phenotype in culture, the method comprising contacting said cells with a serum-free culture medium.

2-24. (canceled)

25. The method of claim 1, wherein the medium comprises a serum replacement composition.

26. The method of claim 25, wherein the serum replacement composition comprises a lipid-rich albumin fraction.

27. The method of claim 26, wherein the lipid-rich albumin fraction comprises a bovine serum albumin preparation.

28. The method of claim 26, wherein the lipid-rich albumin fraction comprises a human serum albumin preparation.

29. The method of claim 1, wherein the serum-free medium comprises one or more amino acids, one or more antioxidants, one or more hormones, or one or more trace element compositions.

30. The method of claim 1, further comprising measuring the expression of DUX4 in said cells.

31. The method of claim 1, wherein said cells assume a myotube morphology.

32. A screening assay comprising:
(a) culturing a cell or population of cells obtained from a subject having, or at risk of having, FSHD under conditions that permit DUX4 expression, and
(b) contacting the cell or population of cells with an agent, and
(c) measuring DUX-4 expression in the cell or population of cells, wherein a decrease in the expression of DUX4 indicates that the agent is a candidate agent for treating FSHD.

33. The method of claim 32, wherein said cells assume a myotube morphology.

34. The method of claim 32, wherein said conditions that permit DUX4 expression comprises culture in serum-free medium comprising a serum replacement composition.

35. The method of claim 34, wherein the serum replacement composition comprises a lipid-rich albumin fraction.

36. The method of claim 35, wherein the lipid-rich albumin fraction comprises bovine serum albumin or human serum albumin.

37. The method of claim 32, wherein the candidate agent reduces the amount of cytopathic lesions, apoptosis, and/or retracted myotubes in the population of cells as compared to a substantially identical cell population cultured under the same conditions but in the absence of the candidate agent.

38. A method for treating FSHD in a subject, the method comprising: administering an inhibitor of DUX4 expression to a subject having, or at risk of having, FSHD, wherein the inhibitor of DUX4 expression is selected from the group consisting of: an activator of the Wnt/β-catenin pathway, a tankyrase inhibitor, a GSK-3β inhibitor, and an activator of DNMT-1, thereby treating FSHD in the subject.

39. The method of claim 38, wherein the activator of the Wnt/β-catenin pathway comprises a recombinant Wnt peptide or polypeptide, or a combination thereof.

40. The method of claim 38, wherein the activator of the Wnt/β-catenin pathway comprises a nucleic acid sequence encoding a recombinant Wnt peptide or polypeptide, or a combination thereof.

41. The method of claim 38, wherein the tankyrase inhibitor comprises Wiki4, XAV-939, IWR or JW55.

42. The method of claim 38, further comprising a step, prior to said administering step, of diagnosing the subject with FSHD.
